(19) United States
(12) Patent Application Publication
(10) Pub. No.: US 2020/0121201 A1
(43) Pub. Date: Apr. 23, 2020

(54) METHOD AND DEVICE FOR THE TIME-RESOLVED MEASUREMENT OF CHARACTERISTIC VARIABLES OF THE CARDIAC FUNCTION

(71) Applicants: Heiko REDTEL, Perleberg (DE); MICRO GIANT DATA TECHNOLOGY (SHENZHEN) CO. LTD., Shenzhen (CN)

(72) Inventor: Holger REDTEL, Perleberg (DE)

(73) Assignees: Heiko REDTEL, Perleberg (DE); MICRO GIANT DATA TECHNOLOGY (SHENZHEN) CO., LTD., Shenzhen (CN)

(21) Appl. No.: 16/493,913

(22) PCT Filed: Mar. 13, 2018
(86) PCT No.: PCT/EP2018/056275

§ 371 Date: Dec. 13, 2019

(30) Foreign Application Priority Data
Mar. 13, 2017 (DE) 102017002334.4
Mar. 13, 2017 (DE) 102017002335.2
Apr. 20, 2017 (DE) 102017003803.1
Jan. 25, 2018 (DE) 102018000574.8
Feb. 21, 2018 (DE) 102018001390.2

(51) Int. Cl. A61B 5/022; A61B 5/02; A61B 5/021; A61B 5/0285; A61B 5/029; A61B 5/00; A61B 5/0402

(52) U.S. Cl. CPC A61B 5/02233; A61B 5/02007; A61B 5/02125; A61B 5/0285; A61B 5/029; A61B 5/681; A61B 5/02422; A61B 5/0402; A61B 2562/0247; A61B 2562/028; A61B 2560/0223; A61B 2562/0261; A61B 2562/0219; A61B 5/02133

(57) ABSTRACT

A time-resolved measurement of blood pressure, arterial elasticity, pulse wave, pulse wave transit time and pulse wave velocity, a cardiac output, and/or changes in cardiac output of a human or animal body, using a pressure sensor unit while being pressed against the skin. The unit is an air and/or gas pressure sensor, and is configured to change at least one electrical conductance and/or resistance when subjected to pressure. The unit has at least two conductor trace arrays, particularly conductor trace networks, and a functional polymer that is compressed when subjected to pressure, and produces and/or alters contact between the conductor trace arrays. Alternatively, the unit has at least two conductive layers with a gap therebetween, and is configured such that the gap becomes compressed when subjected to pressure, and/or such that the capacitance of the assembly composed of the two conductive layers is changed as a result.

The invention relates to the time-resolved measurement of the blood pressure, the arterial elasticity, the pulse wave, the pulse wave transit time, and the pulse wave velocity, and/or the cardiac output and/or changes in the cardiac output of an object, specifically a human or animal body, using pressure sensors for the time-resolved measurement of the energetic pulse wave. The measurement of time-resolved changes in cardiac output requires the measurement of many other parameters of the cardiovascular system. These include the change over time in the blood pressure, the pulse wave transit time, the respiratory rate, and the heart rate. The invention enables a temporal resolution within the millisecond range. Thus, for example, the blood pressure is measurable not only in the form of systolic and diastolic blood pressure values, but also as a continuous wave that indicates the current pressure on the arteries at any point in time, even within a single cardiac pulse.

This temporal precision paired with the ability to take measurements at various locations on the body enables the individual parameters of the cardiovascular system to be determined.

The invention also relates to a pressure sensor unit for the time-resolved measurement of pressure and to a method and a use for pressure measurement in general.

The system according to the invention for the measurement, in particular time-resolved, of blood pressure, arterial elasticity, pulse wave transit time, pulse wave velocity, the pulse wave, and/or of cardiac output and/or changes in cardiac output comprises at least one pressure sensor unit for the time-resolved pressure measurement of the pressure exerted by a pulse wave when said unit is pressed onto the skin, the pressure sensor unit being an air and/or gas pressure sensor and/or being configured to change at least one electrical conductance and/or resistance when subjected to the pressure. In particular, the pressure sensor unit has at least two conductor trace arrays, in particular conductor trace networks, and a functional polymer which is designed to be compressed when subjected to pressure and to produce and/or change contact between the conductor trace arrays.

Wherever a conductance or resistance is mentioned, it should be understood, in particular, as an electrical conductance or electrical resistance.

Alternatively and/or additionally, the pressure sensor unit may have at least two conductive layers with a gap therebetween, and the pressure sensor unit may be configured such that when subjected to pressure, the gap becomes compressed, and/or in particular, the capacitance of the assembly consisting of the two conductive layers is changed as a result. The gap is formed in particular by at least one dielectric. If such a pressure sensor unit and/or an air and/or gas pressure sensor is used as the pressure sensor unit, capacitances are advantageously detected and/or measured instead of conductances and/or resistances, and are used in particular for determining pressure. In general, in place of conductance and/or resistance values, an electrical property can be detected and/or measured and used, in particular, for determining pressure.

The dielectric may be formed by a functional polymer. The functional polymer may be or may contain a dielectric.

In general, the pressure sensor unit may have at least two conductive layers and/or conductor trace arrays, between and/or upon which a volume and/or material is arranged, and the pressure sensor unit may be configured such that when subjected to pressure, the volume and/or material is compressed and/or, in particular, an electrical property of the assembly consisting of the two conductive layers and/or conductor trace arrays changes as a result. The volume and/or material is formed, in particular, by at least one dielectric and/or functional polymer, and/or comprises such a dielectric and/or functional polymer. The volume and/or material, the dielectric, and/or the functional polymer are designed, in particular, to exert a restoring force against compression.

The system has, in particular, an actuator that is configured to press the sensor unit against the skin.

In particular, it has a device for measuring the conductance of the at least one pressure sensor unit. The system is configured, in particular, to measure the conductance and/or pressure with at least a temporal resolution of 5 ms, in particular of 2 ms, more particularly of 1 ms. In particular, the system is configured to use the conductance values to determine pressure values, in particular by means of a conversion and/or correlation, obtained, in particular, by a calibration.

The pressure sensor unit has at least one array of conductor traces and/or conductor trace networks, in particular exposed, and a resistance-conductive and/or conductive polymer, which may be part of the functional polymer, and which is pressed onto the at least one array of conductor traces by an application of pressure. Alternatively or additionally, the pressure sensor unit has at least one non-conductive polymer, which is located between two arrangements of at least one conductor trace each, and which has cavities. In the cavities, the conductor traces are configured, in particular, as exposed. By compressing the non-conductive polymer, also a functional polymer, by means of pressure, contact between the arrangement of the at least one conductor trace is produced, and as the pressure increases, this contact intensifies, so that a conductance that is dependent upon the pressure of the compression results.

The object is also attained by a use of the change in a capacitance, a use of a conductance and/or the change in a resistance and/or in a conductance, and/or a use of a resistance between at least two conductive layers and/or between at least two conductor trace arrays, in particular conductor trace networks, resulting from the compression of a functional polymer and/or dielectric by means of the pressure exerted by a pulse wave when said polymer and/or dielectric is pressed onto the skin above an artery, for the time-resolved measurement of blood pressure, arterial elasticity, pulse wave transit time, pulse wave velocity, the pulse wave, and/or cardiac output and/or changes in cardiac output.

The object is also attained by a pressure sensor unit according to the invention, for example on a gripping system, in particular on a robotic hand, and the use of a pressure sensor unit according to the invention on a gripping system, in particular on a robotic hand, for measuring the pressing force of the gripping system, and by a method for gripping an object with a gripping system having at least one pressure sensor unit according to the invention in such a way that the pressing force of the gripping system on the object acts on the pressure sensor unit, and the measurement of at least one electrical property, in particular conductance, resistance, and/or capacitance, or the change thereof for the time-resolved determination of the pressing force, and also by a method for producing a pressure sensor unit.

The object is also attained by one or by a plurality of pressure sensor unit(s) according to the invention, for example as or in a sensitive sleeve and/or sensitive surface or artificial skin, in particular of a robot, and by the use of one or of a plurality of pressure sensor unit(s) according to the invention as or in a sensitive sleeve and/or sensitive surface or artificial skin, in particular a robotic skin, for measuring the forces on the sensitive sleeve and/or the sensitive surface or artificial skin, and by a method for measuring the forces on a sensitive sleeve and/or a sensitive surface or artificial skin having at least one or a plurality of pressure sensor unit(s) according to the invention, so that forces acting on the sensitive sleeve and/or sensitive surface or artificial skin act on the pressure sensor unit(s), and the measurement of at least one electrical property, in particular conductance, resistance, and/or capacitance, or the change thereof for the time-resolved determination of forces on the skin. The object is also attained by a sensitive sleeve and/or sensitive surface or artificial skin, or in such a sleeve and/or surface or artificial skin, in particular of a robot, having a plurality of pressure sensor units according to the invention.

The pressure sensor unit according to the invention is suitable for such uses, especially due to the ability to detect forces in different measuring ranges with different accuracies using a single pressure sensor unit, and therefore, using such a sensitive sleeve and/or sensitive surface or artificial skin, in particular using the same or structurally identical pressure sensor units, the blood pressure of a living being can be measured, along with significantly higher forces or pressures, for example with the gripping of heavy objects or from 1000 kPa or 10 kg/cm\hat{ }2. In particular, the at least one pressure sensor unit is configured to measure pressures of between 6 kPa and 1000 kPa.

The sensitive sleeve and/or sensitive surface or artificial skin may be embodied as a glove, for example. In particular, the artificial skin has a sensor array composed of a plurality of pressure sensor units. In particular, with a sensor array in this case and generally, the conductor traces and/or at least one conductive layer of a multiplicity of pressure sensor units, in particular of all pressure sensor units, are arranged on a common, integral substrate, and/or the functional polymers and/or structural forms of a multiplicity of pressure sensor units, in particular of all pressure sensor units, are formed together integrally, and in particular are glued to the substrate. The object is also attained by a method for pressure measurement, in particular time-resolved, in particular for the measurement of blood pressure, arterial elasticity, pulse wave transit time, pulse wave velocity, the pulse wave, and/or of cardiac output and/or changes in cardiac output, by way of the change in an electrical property, in particular a capacitance, a resistance, and/or a conductance between at least two conductor trace arrays, in particular conductor trace networks, and/or conductive layers, resulting from the compression of a functional polymer, a gap, a dielectric, a volume, and/or a material, in particular from the pressure exerted by a pulse wave while said functional polymer, gap, dielectric, volume and/or material is pressed against the skin above an artery. In said method, the functional polymer, the gap, the dielectric, the volume, and/or the material is located in particular between and/or on the at least two conductor trace arrays, in particular conductor trace networks, and/or conductive layers.

The conductor trace arrays and/or conductive layers and the functional polymer, the gap, the dielectric, the volume, and/or the material are in particular part of a pressure sensor unit described in this document.

Said unit is pressed against the skin, in particular, at a pressure ranging from 50 to 300 mmHg and/or between 6 kPa and 40 kPa.

The pressure can be transmitted by pressing a pressure sensor unit, in particular configured as described in this document, and/or in particular by pressing an enclosed and pressurized gas volume, in particular air volume, onto the functional polymer and/or onto such a pressure sensor unit. In that case, the pressurization can also be used in particular for pressing said pressure sensor unit and/or said volume on. The pressurized gas has a pressure, in particular, of between 50 and 300 mmHg, and/or between 6 kPa and 40 kPa.

In particular, the conductor trace arrays and the functional polymer are pressed against the skin with varying pressure and the resulting conductance is measured and/or the change in conductance is determined, in particular at least with a temporal resolution of 5 ms, in particular of 2 ms, more particularly of 1 ms, with the varying pressure being increased in particular monotonically and/or continuously, in particular until, with a further increase in the counterpressure and/or applied pressure, the pulse wave is not able to generate an increase in the measured pressure beyond the maximum measured pressure, and with the applied pressure being applied in particular by the inflation of an air bag or by means of some other actuator.

An air bag is understood, in particular, as a device having an enclosed volume, in particular with a flexible outer covering, for example, a pressure cushion. An air bag is configured, in particular, to be supplied with gas and thereby pressurized, in particular with an expansion of its volume. More particularly, the air bag is configured such that, once it has been pressurized, it will apply pressure to an object, for example an arm, which is encircled by the air bag, or to an encircled object, for example an arm, which is encircled by an encompassing device, and in particular is encircled by the air bag, the air bag being arranged, in particular, between the encircled object and the encompassing device, in particular without itself encompassing the encompassed volume. In particular, when a pressure sensor unit is used that is not designed to be pressed against the skin, and/or that is or will be positioned on the air bag, in the air bag, and/or in a volume that is fluidically connected to the air bag, and/or adjacent to such a volume or to the air bag, in particular a means for calibration is provided and/or in particular a calibration is performed, in order to compensate for the influence, for example attenuation, by the coupling and/or the air bag. For this purpose, in particular, a blood pressure measurement is first performed using other means, for example a previously known blood pressure measurement and/or using previously known means and/or methods for measuring blood pressure, and/or other means for measuring blood pressure, such as a microphone and/or stethoscope for traditional blood pressure measurement, are included in the system or are used. In that case, another pressure sensor, which may be included in the system, can be used to perform a blood pressure measurement. In parallel and/or in a close temporal relation of no more than 10 seconds from said blood pressure measurement, in particular at least the air pressure or gas pressure, capacitance, conductance, and/or resistance, in particular of the at least one pressure sensor unit, is measured, in particular with at least a temporal resolution of 2 ms, in particular 1 ms, and based upon the blood pressure measurements using the other means and/or previously known means and/or methods for measuring blood pressure, the measurement of air pressure or gas pressure, capacitance, conductance, and/or resistance is calibrated, in order to then enable and/or carry out blood pressure measurements by means of the at least one pressure sensor unit.

Blood pressure measuring means and/or methods function in particular by increasing the pressure in the air pressure cuff and measuring the pressure in the air pressure cuff or in a volume that is fluidically connected thereto. From a certain pressure in the air pressure cuff, the pulse wave causes a fluctuation in the measured pressure, which decreases again with a further increase in the pressure in the air pressure cuff. The pattern of these fluctuations shows a progression over time. In the prior art, the diastolic and/or systolic blood pressure is derived from this progression over time and/or from the envelopes of the fluctuations. However, such a system can also be used according to the invention for measuring the pulse wave or for determining blood pressure at one pulse wave. For this purpose, in particular at least one measurement is carried out according to the previously known method and is then used to calibrate the measured values obtained from the measurement of air pressure or gas pressure, capacitance, conductance, and/or resistance, to enable the pressure of the pulse wave to be derived directly from these measured values.

Systems, methods, and/or uses according to the invention are thus configured and/or embodied, in particular, such that values for systolic and/or diastolic blood pressure, arterial elasticity, pulse wave pressure, pulse wave transit time, and pulse wave velocity, and/or for cardiac output and/or changes in cardiac output are each related to one pulse wave rather than being based upon a plurality of pulse waves, as is the case, for example, with the described previously known derivation from the envelopes.

The at least one pressure sensor unit can be pressed by the air bag, for example, onto the skin above an artery. Alternatively, for example, the impact of the pulse wave on the pressurized gas contained in the air bag can be transmitted through the air bag. For this purpose, the pressure of the gas in the air bag is, in particular, between 50 and 300 mmHg and/or between 6 kPa and 40 kPa and/or is built up in particular by the actuator. The pressure sensor unit can thus also be positioned such that it can detect the pressure fluctuations in the gas of the air bag.

In particular, from the conductance values and/or the changes thereof, the pressure and/or a pressure is determined, and/or a change in the pressure is determined.

The systolic blood pressure is assumed, in particular, to be the pressure at which or from which, as the counterpressure and/or applied pressure continues to increase, the pulse wave does not produce an increase in the measured pressure beyond the maximum measured pressure, and/or the diastolic blood pressure is assumed to be the pressure that corresponds to the minima of the measured values of a pulse wave when the chosen counterpressure and/or applied pressure is the pressure, or higher than the pressure, at which, as the counterpressure and/or applied pressure increases, the maximum measured pressure does not increase any further.

One particular advantage of the invention is that the values, such as systolic blood pressure and diastolic blood pressure, can be determined non-invasively from a single pulse wave, which is preferable, and thus said values are also in direct physical and physiological correlation.

In particular, the pressure of the applied pressure is reduced subsequently and/or after determination of a systolic blood pressure, in particular to a value ranging from the determined diastolic to the determined systolic blood pressure, and/or up to 1.5 times, in particular 1.3 times the systolic blood pressure of the pulse wave pressure in the systole and/or the systolic blood pressure, and/or from 60 to 120 mmHg, in particular from 60 to 90% of the systolic pressure of the pulse wave in the systole, in particular at the measurement site, and/or to a value low enough that the measurement signal, in particular the conductance, resistance, or capacitance, of the at least one pressure sensor unit still has a variation with the cardiac pulse, which is typically possible at up to 80% of the pressure of the diastolic blood pressure.

In particular, the pressure of the applied pressure is reduced or removed subsequently, and/or after determination of a systolic blood pressure, and/or with a known first systolic blood pressure and/or first conductance value of the at least one pressure sensor unit when subjected to the first systolic blood pressure, the counterpressure and/or applied pressure is reduced to less than 1.1 times the first systolic blood pressure or less than the first systolic blood pressure or to the mean value between diastolic and systolic blood pressure, or is removed, and the ratios between the conductances that are then measured and the first conductances and/or the ratios between the pressures associated with the then measured conductances and the first systolic blood pressure are used as a factor for determining the current blood pressure, the current arterial elasticity, the current pulse wave transit time, the current pulse wave velocity, the current pulse wave and/or the current cardiac volume and/or the current change in cardiac volume, from the first systolic blood pressure.

The method is carried out, in particular, by means of a system according to the invention.

The object is also attained by using the change in an electrical property, in particular in a capacitance, a conductance, and/or an electrical property, in particular a capacitance, a resistance, and/or a conductance between at least two conductive layers and/or between at least two conductor trace arrays, in particular conductor trace networks, resulting from the compression of a functional polymer, a gap, a dielectric, a volume, and/or a material by the pressure exerted by a pulse wave while said functional polymer, gap, dielectric, volume and/or material is pressed against the skin above an artery, for the time-resolved measurement of blood pressure, arterial elasticity, pulse wave transit time, pulse wave velocity, the pulse wave, and/or cardiac output and/or changes in cardiac output.

The object is also attained by a method for retrofitting previously known air pressure measuring systems that have an air pressure cuff, a device for pressurizing the air pressure cuff, and an air and/or gas pressure sensor, in which the air pressure measuring system is provided with an analysis device that is configured to carry out a method according to the invention, in particular in a configuration described as advantageous, and/or in which an analysis device already included is modified such that it is configured to carry out a method according to the invention, in particular in a configuration described as advantageous.

The advantageous embodiments with respect to the method, the pressure sensor unit, the system, and/or the use can be transferred to the method, the pressure sensor unit, the system, and/or the use.

In general, in place of one or more conductances, one or more resistances may also be used. In that case, maxima and minima must be exchanged accordingly, since conductance is the inverse value of resistance.

A resistance-conductive and/or conductive polymer can be produced in two ways, in particular: For one, the polymer can be chemically structured such that it is intrinsically conductive;

this can be achieved by conjugated double bonds between the carbon atoms in the polymer chains, for example. This type of polymer is a more recent and less commonly used class of materials than normal polymers. It is therefore expensive, and the variability of the properties of current variants is insufficient for sensor construction.

Conductive materials can also be incorporated into a conventional polymer, for example. These may include carbon black, graphite, or metal particles, for example, in particular within the range of a few nanometers. An ink, for example from Loctite, may be used as the resistance-conductive and/or conductive polymer. Such an ink typically consists of a dissolved thermoplastic, which is mixed with electrically conductive particles, such as graphite. These inks have optimal electrical properties, but their abrasion resistance is poor, making the lifespan of a sensor short. A thermoplastic is a polymer in which the individual polymer strands are loose, similar to spaghetti. Abrasion resistance can be improved by crosslinking the individual polymer strands, which makes the material rather rubbery or changes it to an elastomer.

Crosslinking can be accomplished during production by introducing various catalysts, for example vulcanizers such as sulfur, into the ink and/or the resistance-conductive and/or conductive polymer. Aftermarket crosslinking is complicated and usually costly. For instance, free radicals can be generated in the resistance-conductive and/or conductive polymer. These engage the polymer chains and create reactive sites that react with other chains to produce a network. These radicals can be generated using either radiation or chemical substances. For the radiation, typically electron beams are used. With chemical treatment, peroxides are introduced into the polymer, which gradually break down and release free radicals.

Since the conductive polymer is typically a thin layer, chemical crosslinking is an option. Liquid peroxides can diffuse into the material and can induce chemical reactions in the material (but near the surface). With a given exposure time, greater crosslinking and thus greater stability in the surface material can be produced.

Studies to improve this process have shown that even hydrogen peroxide produces a positive effect. This is advantageous particularly because it is a cheap, relatively harmless, and relatively environmentally neutral chemical as compared with other peroxides. However, it requires longer exposure times.

Greater reactivity of hydrogen peroxide can be achieved in two ways. For one, during the exposure period the temperature can be increased. For another, a solvent can be used to swell the polymer, enabling increased diffusion into the material. In the first method, temperatures of 120-160° C. are typically used. However, the melting points of many thermoplastics also lie within this temperature range, making close temperature monitoring essential. The second method is also problematic because mixtures of peroxides and solvents form the basis for many explosives.

Time-resolved and/or temporally resolved means, in particular, that the measurement is carried out with a temporal resolution, or the system is configured for measurement with a temporal resolution that allows the pressure maxima and pressure minima of a human pulse wave to be detected, in particular with an inaccuracy of at most 10% with respect to the pressure and/or the time of the pressure maxima and/or pressure minima, with respect to the time, in particular within a pulse wave, and/or an accuracy of 10 ms or better. In particular, measurement is performed, and/or the system is configured for measurement, in particular, of at least one electrical conductance, resistance, and/or at least one capacitance, with a repetition rate of at least 100 Hz, in particular at least 500 Hz, more particularly at least 800 Hz, in particular at least 1 kHz.

Ascertaining the parameters of the cardiovascular system is based upon an analysis of the measured values of the pulsatile pressure wave in the arteries coming from the heart.

Since an accuracy or a data acquisition rate of the pulsatile pressure wave of one millisecond is achieved, in particular, the measured values of the pulsatile pressure wave, also referred to in their temporal sequence as the measured value wave, can be analyzed for their minima and maxima. When applied correctly, the values of these minima and maxima correspond to the values for the value of the diastolic or systolic portion of the traditional blood pressure value. Moreover, from the interval of time between the minima and the maxima, the current cardiac pulse can be determined, specifically from pulse to pulse, which allows the pulse wave variability to be calculated. In particular, the simultaneous determination of cardiac pulse and blood pressure enables the cardiac output to be calculated.

An assembly or system according to the invention can also comprise multiple pressure sensor units. This allows the pulse wave transit time to be measured by performing measurements at different measurement sites.

The pulse wave transit time can be determined by a plurality of pressure sensor units, at least two, and/or at least one pressure sensor unit and one device for measuring the pressure wave and/or the pulse recording the pulsatile pressure wave and/or the pulse at at least two measurement sites on the body. The time interval between two maxima and/or correlating events that are attributed to the same cardiac pulse is used to determine the pulse wave transit time between the measurement sites, and in particular is used to determine the pulse wave velocity, assuming the distance between the measurement sites and/or the distance of the measurement site from the heart is known.

The pulse wave transit time can also be determined by analyzing the measured values of the pulsatile pressure wave, in which the reflected wave is identified and the interval of time between the reflected wave and the initial wave is determined as the pulse wave transit time.

When the heart pumps blood out, the pulse wave first enters the aortic arch, after which that artery branches into smaller arteries. Due to the differences in diameter before and after branching, reflection occurs at each branch. The greatest reflection in terms of amplitude occurs in the smallest arteries, and this can be detected in the pressure wave.

The pulse wave velocity can be determined from the pulse wave transit time, assuming the distance between the measurement sites and/or the distance of the measurement site from the heart is known.

The elasticity of the arteries can also be determined from the pulse wave velocity, e.g., using the Moens-Korteweg equation.

Furthermore, due to the temporal resolution of the data acquisition, also called the data acquisition rate, the sensors do not need to be spaced particularly far apart from one another, and thus, a system having a plurality of pressure sensor units, which is perceived by the user as a single unit, may be used. This enables a very simple and rapid measurement of these parameters, a process which would require lengthy preparation times and a large number of very different sensors with the measuring instruments currently in use.

As will be described below, a measurement according to the invention is carried out in particular as follows. The system or the pressure sensor unit is positioned at a suitable site, in particular a site above an artery, which may be a point on the wrist, for example, after which pressure is slowly applied. This pressing of said unit with an applied pressure or the adjustment of counterpressure may be implemented either by human action or by means of an autonomous actuator. At the same time, measured values from the pressure sensor unit, in particular conductances from which a pressure can be derived and which are influenced by the pulsatile pressure wave of the artery, are detected. When the counterpressure or applied pressure is increased, in particular from a level of 60 mmHg or less, the maxima of the detected pressure and/or of the measured value wave and/or of the measured values also increase. Beyond a certain applied pressure or counterpressure, no further increase in the maxima is observed. The pressure value of a maximum pressure and/or the pressure associated with a maximum conductance value is the systolic blood pressure value. At the lowest applied pressure and/or counterpressure at which no further increase in the maxima of the measured value wave and/or of the measured values is observed, the pressure value of a minimum of the measured value wave and/or the pressure associated with a minimum corresponds to the diastolic blood pressure.

In each case a maximum corresponds to a systolic blood pressure of a pulse wave and a minimum corresponds to a diastolic blood pressure of a pulse wave.

In particular, if this counterpressure or applied pressure, beyond which, with a further increase, no further increase in the maxima of the measured value wave and/or of the measured values is observed, persists, a continuous measurement can be carried out, in which each pressure value of a pressure maximum and/or the pressure associated with each maximum conductance value represents the systolic blood pressure value of the respective pulse wave, and/or each pressure value of a minimum of the measured value wave and/or each pressure associated with a minimum represents the diastolic blood pressure of a pulse wave.

Particularly advantageously, the method is carried out and/or the system is configured such that at least one pressure value, in particular at least two pressure values, of at least every twentieth, in particular of at least every tenth, more particularly of every second or of each pulse wave, in particular of at least 50, more particularly of at least 500 successive pulse waves, are determined and/or displayed. In particular, 5 to 20 pressure values and/or pressure values for 5 to 20 pulse waves are displayed simultaneously.

Particularly advantageously, the method is carried out and/or the system is configured such that measurements are performed continuously, i.e., in particular at least every twentieth, more particularly at least every tenth, in particular every second, or each pulse wave of at least 500 successive pulse waves are measured, and/or from at least every twentieth, in particular from at least every tenth, more particularly every second, or every pulse wave of at least 500 successive pulse waves, at least one blood pressure value, one arterial elasticity value, one pulse wave transit time value, one pulse wave velocity value, and/or one value for cardiac output and/or one value for changes in cardiac output is determined and/or displayed. In particular, 5 to 20 values and/or values for 5 to 20 pulse waves are displayed simultaneously.

Particularly advantageously, the method is carried out such that blood pressure, arterial elasticity, pulse wave transit time, pulse wave velocity, the pulse wave, and/or cardiac output and/or changes in cardiac output, in particular the pressure curve of the pulse wave, is measured in least two, in particular four, extremities, and the measured values obtained from the measurements at the extremities are compared, in particular those that are attributed to the same heartbeat.

Particularly advantageously, the system is configured to measure blood pressure, arterial elasticity, pulse wave transit time, pulse wave velocity, the pulse wave, and/or cardiac output and/or changes in cardiac output, in particular the pressure curve of the pulse wave, in least two, in particular four, extremities, the system having, in particular, at least one pressure sensor unit per extremity, and is configured to compare the measured values obtained from the measurements taken at the extremities, in particular those measured values that are attributed to the same heartbeat.

In this case, measurements are taken in particular on two of the same extremities, except for the left/right arrangement, in particular on the same blood vessels, in particular arteries, in particular at identical locations on opposite sides of the body.

The maxima and/or minima are in particular local maxima and minima.

Calibrations described here are not to be performed by the user, and can instead be automated or performed during manufacturing.

The pressure sensor units described in this patent are not influenced by acceleration forces.

The reason for the optional use of an acceleration sensor in this patent is based on the fact that the blood pressure value is to be measured at different locations in the body and also at different heights relative to the hydrostatic indifference point (HIP) (changes in the height of a measurement site relative to the HIP are triggered, e.g., on the arm by an arm movement). If the current height relative to the HIP is known, a value for blood pressure at the HIP can also be determined in a movement, even though the measurement site is located, e.g., on the arm.

If a sensor array having a plurality of pressure sensor units, in particular adjacent to one another, is used, the sensor that is in the most optimal position above an artery may be selected; this increases the ease of use, since a complex positioning of the sensor module is not necessary. A sensor array can also be used to determine the pulse wave transit time at the measurement site by analyzing the measured values of the pulsatile pressure wave from at least two pressure sensor units of the sensor array. In particular, at least two maxima of the measured values of the pulse wave, which in particular are attributed to the same heartbeat, are used for this purpose. If the distance between the at least two pressure sensor units is known, in particular, a pulse wave transit time can then be calculated, in particular with the aid of the analysis unit.

In another inventive assembly according to this patent, a plurality of sensors may be organized separately from one another so that one sensor can be attached near the heart, for example, and another can be attached at a suitable location on the wrist, for example. In this case, the analysis of the measured value wave of the pulsatile pressure wave enables the pulse wave transit time from the heart to the wrist to be calculated.

The invention can be implemented with a minimal sensor size and requires no invasive procedures in the body.

### BRIEF DESCRIPTION OF THE DRAWINGS

FIG. 1 shows an exemplary illustration of the measurement method.

FIG. 2 shows an exemplary diagram of a conventional wristband.

FIG. 3 shows, by way of example, a cross-section of one possible embodiment of the assembly of the invention for use on the wristband as an attachment.

FIG. 4 shows an exemplary electrical circuit comprising multiple pressure sensor units in the crossover circuit.

FIG. 5 shows exemplary raw data from an assembly according to the invention.

FIG. 6 shows a schematic and exemplary illustration of the measurement of pulse wave velocity by means of a sensor array.

FIG. 7 shows examples of possible embodiments of the inventive configuration of the conductor trace arrays.

FIG. 8 shows a conductor trace array having three conductor traces.

FIG. 9 shows a cross-section of two pressure sensor units arranged side by side and configured as VRS sensors.

FIG. 10 shows a cross-section of two pressure sensor units arranged side by side.

FIG. 11 shows an exemplary configuration of a measuring system for measuring conductances, and from these, pressures.

FIG. 12 is a schematic illustration showing one possible embodiment of the device according to the invention.

FIG. 13 is a schematic illustration showing one possible embodiment of the device according to the invention.

FIG. 14 shows a cross-section of a pressure sensor unit according to the invention.

FIG. 15 shows a cross-section through another embodiment of a pressure sensor unit according to the invention.

When pressure sensor units that measure by an application of force are used, the sensor can be placed directly on the skin; see FIG. 1, (O). Preferably, the size of the pressure sensor unit, in particular its pressure-sensitive surface area and/or its contact surface area on the skin, is no larger than a cherry pit and/or is less than 15 mm, in particular less than 10 mm, more particularly less than or equal to 5 mm in diameter, in particular for performing a blood pressure measurement on the skin.

The operating principle of the invention is based upon the operating principle of the traditional method for blood pressure measurement, the Riva Rocci method. However, the invention expands upon this method to include the temporal resolution of blood pressure value determination and can therefore also be used for continuous long-term measurement. In addition, measurement is less painful due to the small sensor size. This is advantageous especially for continuous long-term measurements.

The pressure sensor units and an analysis unit may be used alone. Advantageously, however, the pressure sensor unit is integrated with an analysis unit and/or a power unit and/or a wireless unit in a system and/or a device and/or a piece of clothing, and/or is designed as an attachment. Suitable pieces of clothing include wristbands, ankle straps, shoes, rings, and ear clips. The inventive assembly can also be fastened onto the body with the aid of specially designed straps. If the inventive assembly and/or the system is designed as an attachment, it can be fastened onto the body by attachment to a conventional wristband or, for example, by attachment to or insertion into the shoe/tongue (instep).

Advantageously, the inventive assembly and/or the system can also be expanded to include an actuator, which can exert a base pressure or applied pressure onto the pressure sensor unit, and/or which presses and/or forces the pressure sensor unit and/or the sensor array against the skin with a base pressure or applied pressure. However, the inventive assembly can also be operated without an actuator.

As will be further described in this specification, the inventive assembly can be attached to the body in such a way that pressure can be exerted onto the body by the pressure sensor unit.

Especially advantageous for this are locations on the body where the pulse of the arterial system can be detected. These include positions on the wrists or positions on the insteps, for example.

Theoretically, some type of technology, such as that of an FSR sensor (Force Sensing Resistor), can be used as a pressure sensor unit for measuring blood pressure. This technology is described in a patent held by Interlink, and is accessible to those skilled in the art on the Internet in multiple publications by Interlink. This described pressure sensor is also produced by Interlink and has been available commercially for many years. The pressure sensor is available in various sizes. The functioning of the FSR sensors included under this designation involves an electrically conductive paste or substance being applied to the substrate material, but above the electric leads.

In most cases, however, a pressure-sensitive and resistance-conductive film is used, which is applied with a substrate to the electric conductor traces, and these are connected to one another by means of a double-sided adhesive layer. The necessary information about this is available to those skilled in the art. However, the offerings of “Interlink” with respect to FSR sensors are limited to a pressure-sensitive film that changes its resistance-conductivity when subjected to pressures, or weights.

FSR sensor technology was not developed for an accurate and consistent measurement of pressure. Severe fluctuations in the continuous recording of measured values lead to inaccuracies in the measurement of weight and thus also to unsuitability in medical applications.

Conventional sensors can be calibrated by subjecting the sensor to a known pressure. This may be a motorized wristband, for example, which adjusts to a known pressure by way of a defined constriction. This pressure of the wristband can be determined, e.g., by strain gauges (these are not suitable for the actual measurement because the temporal resolution is too low).

Advantageously, however, calibration is carried out with the aid of a vibration motor, in particular contained in the system, and/or by a variation of the applied pressure by means of the vibration motor. Said vibration motor can apply a defined pressure to the sensor by means of a suitable electrical circuit, which is known to those skilled in the art, and can thereby perform a calibration. A pressure sensor unit may be attached to the inner side of a band, for example. The vibration motor can be located between sensor and band.

When the motor vibrates, it pushes the band and sensor apart, or if the band is worn on the arm, this spreading apart leads to a change in the pressure applied by the pressure sensor unit onto the skin. Thus, a vibration motor is located in particular between encompassing device and pressure sensor unit.

Advantageously, however, a one-time factory calibration is used, in particular.

As a solution, the invention prefers small structural forms that are specific to their application for blood pressure measurement, in particular in the form of an elastic molded article.

Advantageously, the sensor should meet the following requirements:
Flexible shape, so that the sensor can adapt to the respective measurement site on the body, and/or
Soft design of the sensor to prevent injuries, and/or
A shape that adapts to the body to maximize coverage of the sensor, and/or
A small sensor size, advantageously with a diameter of 5 mm or less. However, larger and smaller sizes are also possible, and/or
A consistent measurement quality, if possible without calibration. A continuous measurement over a period of two weeks or more must at least be guaranteed, and/or
A measuring range that covers the anticipated blood pressure range; this should be at least 40 mmHg or 5 kPa and/or up to 300 mmHg or 40 kPa, and/or
Should have a pressure resolution of 0.5 mmHg or less, and/or
Should have a temporal resolution of 1 ms or less, and/or
The sensor should attenuate the signal as little as possible, and/or
Should be weather and moisture resistant. This also includes resistance to perspiration, and/or the measurement process must be possible with low power consumption, in order to enable mobile measurement, e.g., battery operation, and/or
Measurement must be possible with few other components in addition to the actual sensor, in order to enable a small structural form.

The invention therefore uses new types of sensors, in particular, as the pressure sensor unit, which will be presented below; however, the use of an FSR sensor or piezo sensor as the pressure sensor unit is also possible.

Advantageously, an SRS sensor (switchable resistive sensor) can be used to measure cardiac output. This type of sensor has multiple measuring ranges (at least two different measuring ranges). This makes it possible, for example, to collectively cover one large measuring range given by the sum of the individual measuring ranges, and/or to cover one or more measuring ranges with different degrees of accuracy. Particular advantageously, the measuring ranges overlap at least partially and/or have different sizes and/or ranges. Different ranges with the same absolute changes in conductance over the respective measurement ranges and with the same measuring accuracy result, in particular, with different resolutions of the measurement, in particular of the pressure. Furthermore, the individual measuring ranges are independent of one another, i.e., blood pressure can be measured in the different measuring ranges simultaneously, and thus in particular with different accuracies and/or in different measuring ranges and/or pressurization ranges. Advantageously, the pressurization ranges overlap.

As compared with other types of sensors, e.g., FSR sensors (Force Sensing Resistor) from Interlink or piezoelectric sensors, a greater overall measuring range can be covered. This is advantageous, in particular, because when the body moves, the blood pressure signal, which changes over time, varies based upon the height of the sensor, the measuring point, e.g. on the extremities, in relation to the HIP. Variations in the blood pressure signal may have several causes. In addition to movements of the body, changes in temperature or medications, for example, may lead to a sudden change in the blood pressure signal.

Unlike FSR sensors, SRS sensors have at least three conductor trace arrays or conductor trace networks or conductive layers, whereas FSR sensors have only two conductor trace arrays or networks. For reading out the conductances and/or resistances, VSR sensors likewise have, in particular, only two conductor trace arrays or conductor trace networks. But at least one additional conductor trace array or conductor trace network or electrode is necessary for induction.

A conductor trace array, a conductor trace network, or an arrangement of a conductor trace network has at least one conductor section, in particular a plurality of conductor sections, in particular branched and/or planar and/or finger-like and/or wound, and it may have loops and/or openings and/or may be labyrinthine in configuration.

The conductor trace arrays or conductor trace networks are interconnected, in particular, and/or in particular have parallel conductor trace sections.

The conductor traces, conductor trace sections, or conductive layers may be metallic and/or doped semiconductors, for example, and/or may be made of a conductive polymer.

Conductive layers are configured, in particular, as planar, without cavities or recesses.

Typically, a conductive polymer has higher resistance than a metallic conductor, such as copper. Therefore, only what is absolutely necessary should be pressed or embodied using the conductive polymer, as otherwise either the anticipated power consumption will be greater or the signal quality may suffer. In the case of digital lines, if conductors made of polymer are too long, signal transmission may be adversely affected.

In addition to resistance in the conductor, conductive polymers also have increased contact resistance. This means that simply pressing a metallic conductor onto a polymer conductor (e.g., by crimping) typically will not produce effective contact. However, in most cases a transition point from polymer to metallic conductors is unavoidable.

Since polymers, especially for use in 3D printing, are fusible, however, another variant of the connection is produced by heating the metallic conductor and pressing it into the polymer conductor. This causes the polymer conductor to melt in localized areas, and the metallic conductor sinks in. The result after cooling is an electrical contact. The metallic conductor can thus be embedded in the polymer.

To improve the mechanical and electrical connection, the end of the metallic conductor may be molded in the form of a net or one or more eyelets.

Since the SRS sensor is able to detect the signal of the pulsatile pressure wave with multiple measuring ranges simultaneously by using the at least three conductor trace arrays or conductive layers, the best measuring range can be used without switching the readout electronics.

A further advantageous sensor is the VRS (Variable Resistive Sensor). This is a sensor whose measuring range can be changed by electrical induction. In this case, the measuring range is changed by induction, in particular, in the functional polymer of the sensor or of the pressure sensor unit. Thus, a large measuring range can be covered with this type of sensor as well. With VRS sensors, the conductor trace array for ascertaining conductances and/or resistances can be selected, as with FSR sensors, but the sensitivity of the polymer is additionally changed by electrical induction. The measuring range is chosen is based upon the degree of electrical induction.

In general, the conductor trace arrays, conductor trace networks, and/or at least one conductive layer are arranged in particular on an electrically insulating substrate.

When this type of sensor is used, there are basically two measuring methods. In one, a fixed measuring range that is changed only as needed can be set, and the signal of the pulsatile pressure wave is measured directly. In the other, the pressure can also be measured indirectly by altering the electrical induction to select the measuring range until a defined signal results. The actual measured value in this case is the electrical induction setting. The direct measurement enables a faster generation of measurements, whereas the indirect measurement enables more accurate measurements.

The basic configuration of the conductor traces of a VRS sensor is similar, in particular, to the configuration of an SRS sensor with two measuring ranges, i.e. with three conductor trace networks. However, only two conductor trace networks are used, in particular, between which the resistance is measured and is used as a measured value. In particular, a third conductor trace network, an electrode, and/or a third conductive layer are used together with an additional (fourth) conductive layer on the opposite side (as viewed from the conductor trace networks) of the functional polymer. Between the additional (fourth) conductive layer and the third conductor trace network, the electrode, and/or the third conductive layer a voltage is applied, in particular in order to induce a voltage and/or to alter the properties of the functional polymer.

The functional polymer has the property of reacting to this applied voltage. The reaction consists in a change in the measuring range. This involves two mechanisms of action that can be exploited individually or together. Firstly, the electrical conductivity of the polymer can be changed. And secondly, its mechanical properties can be changed.

One example of a functional polymer that can alter its electrical properties consists of a non-conductive soft base material into which elongated electrically conductive particles are incorporated. The particles also have an electric dipole moment.

When no voltage is applied, these particles are randomly oriented. Applying a voltage causes the particles to become oriented along their dipole moment. The average angle of alignment of the particles to the field of the applied voltage is dependent upon the magnitude of the applied voltage. The electrical properties perpendicular to the voltage field, i.e., in the direction of measurement of the resistance of the sensor, are dependent upon the distance between the conductive particles in the direction of measurement.

If rod-shaped particles are used, which align perpendicular to the sensor surface when a voltage is applied, the distance between the particles in the direction of measurement (parallel to the sensor surface) increases as the alignment and the internal resistance of the polymer increase. To achieve the same resistance between the first two conductor trace networks, the polymer must then be pressed more firmly against the conductor traces so that less contact resistance results to outweigh the now greater internal resistance of the polymer. The measuring range of the pressure application is shifted upward.

Functional polymers that can alter their mechanical properties by the application of an electric voltage are referred to collectively as electroactive polymers. The polymer Nafion may be used, for example. This polymer deforms with application of a voltage of 1 to 5V.

A planar and deformable electrical contact can be applied to both sides of the electroactive polymer. A layer of electrically conductive polymer, in particular, is applied to one of these electrical contacts, with a non-conductive layer advantageously being applied between the electrical contact and the conductive polymer.

A voltage applied to the electrical contacts causes a deformation of the polymer. The electroactive polymer may be designed as decreasing in thickness concentrically outward, whereby a hemispherical deformation is triggered. Since movement of the polymer is restricted, the polymer thus deformed is pressed against the conductor traces.

When a voltage is applied, the electroactive polymer is pressed against the conductor traces, and a lower external load is required to obtain the same measured value as is obtained without deformation of the polymer. The measuring range is shifted to smaller loads.

The sensor types themselves present an electrical resistance, and change their resistance value when subjected to force or pressure. The SRS has different resistances for different measuring ranges, while the VRS sensor, in particular, has only one resistance. VRS sensors and SRS sensors may be combined in one pressure sensor unit, in that the measuring range of an SRS sensor is changed by induction in the functional polymer of the SRS sensor or the pressure sensor unit.

The conductor traces and/or networks and/or sections are isolated from one another, in particular.

The sensors described herein are based on the concept that a polymer, including a functional polymer, which is resistance-conductive and/or conductive and/or which has a resistance-conductive and/or conductive surface section and/or a resistance-conductive and/or conductive surface, is pressed by an application of force against an array of conductor traces, which in particular are exposed. Said conductor traces are not meant to be fully exposed, in particular, but should be exposed enough that the functional polymer can contact them electrically by touching them.

Therefore, in practice, both the array of the conductor traces and the properties of the polymer are adapted to the application.

With the SRS sensor, in particular a plurality of conductor traces, conductor sections, conductor arrays, and/or conductor trace networks mesh with one another. The number of conductor trace arrays or conductor trace networks is determined by the number of measuring ranges and is equal to the number of measuring ranges plus one.

The properties can be adapted by adapting the conductor trace networks and/or conductor trace arrays of the pressure sensor unit to the measuring requirements, which may be achieved by adapting the distances between the conductor traces and/or conductor trace networks, arrays, and/or sections, the widths of the conductor traces and/or conductor trace networks, arrays, and/or sections relative to one another, and the area coverage of the conductor traces and/or conductor trace networks, arrays, and/or sections. Properties can further be adjusted by the selective lacquering of individual areas.

For a quick and inexpensive adjustment of these parameters, it has proven advantageous to insert a further non-conductive polymer layer.

The process of adjusting the conductor traces thus involves first fabricating a sensor that, according to experience, roughly meets the requirements. An additional polymer layer can then be inserted between the conductor traces and the resistance-conductive polymer. This additional polymer layer is non-conductive.

The non-conductive polymer layer is provided with cavities, strips, or other types of recesses.

The surface area and the precise extent of the recessed areas are then varied until the desired measuring range is found. This is advantageous especially since this additional polymer layer is inexpensive and can be quickly replaced.

Once an optimal surface area ratio is identified, a lacquer coating that corresponds to the non-conductive polymer layer is used in fabricating the sensors.

The relationship between pressurization and conductance and/or the inverse value of the resistance value of the pressure sensor unit is in particular linear, in particular in each of the measuring ranges.

The challenge is to find a layout of the conductor traces and/or sections in which each of the individual conductor trace networks covers every point on the surface area, but there is also a distance between the networks. This is not possible. The closest approximation to this is preferred.

The distance between the conductor trace networks determines the pressure resolution of the pressure sensor unit. This resolution results from the interaction with the mechanical properties of the polymer. If an identical polymer is used: the shorter the distance, the higher the resolution. However, this runs counter to the maximum measuring range. There must be a trade-off between resolution and maximum measuring range.

The coverage of the surface area by each of the individual conductor trace networks determines the accuracy of the sensor. When the polymer is pressed against the conductor traces, the polymer first touches these at one point, in particular. If only the conductor traces of a conductor trace network are present at said point, no electrical contact will be produced between the different conductor trace networks, and the application of pressure will not result in a change in the conductance.

At higher pressure loads, electrical contact already exists between the conductor trace networks, and this contact is improved when the polymer comes in contact with a larger surface area on the conductor traces. If, as the pressure load increases, the contact area increases beyond an area that either is covered by only one conductor trace network or is not covered by any conductor trace network, there will be no change in the measured value and this load range is not detectable. The accuracy results from the size of these blind load ranges.

One possible configuration that has proven suitable for many applications is provided by alternating conductor traces of equal width or thickness, with a spacing between the conductor traces that corresponds to half the width or thickness.

The operating principle of a polymer pressure sensor is based upon the resistance-conductive and/or conductive polymer being pressed with more or less force against the conductor traces, depending on the load, and thus producing a (resistive) electrical contact between two conductor trace networks. The electrical resistance and conductance vary dependent upon the load of the sensor.

In general, there are various basic approaches for optimizing the conductor trace layout. For example, the width and arrangement of the conductor traces may be varied to produce a different surface area coverage with conductor traces.

For example, a lacquer coating may be applied. This lacquer coating covers different regions of the conductor traces.

Another lacquer coating option involves partially coating the surface area on which the conductor traces are located, to partially cover the conductor traces. The more the conductor traces are covered, the greater load is required to produce a good electrical contact. The maximum measuring range increases. One example of this is a hemispherical or spherical cap-shaped polymer. When this polymer is pressed against the conductor traces, the contact surface area increases with the application of pressure. In this process, the surface area increases concentrically as the pressure load increases. The larger the surface area, the better the electrical contact and thus the measured value.

If concentric rings were then to be applied as a lacquer coating, increasing the load would produce load areas in which the contact area is increased only over the lacquer coated areas, i.e., in these load areas the measured value would remain constant and the sensor would not recognize this measuring range. This phenomenon should be avoided, and therefore, with a hemispherical or spherical cap-shaped polymer, star-shaped markings are better.

A suitable lacquer coating can also be used to increase the lifespan of a sensor. Conventional methods for producing printed circuit boards do not result in an ideally smooth surface, but one in which the conductor traces stand out. The height of such conductor traces is typically 35 or 50 μm;

other heights can also be produced, but a height of 0 μm is not possible. When a load is applied to the sensor, the conductor traces are pressed into the polymer. This leads to increased stress at these points in the polymer, and abrasion can occur; measurement quality decreases. To prevent this, an ideally smooth surface would be desirable.

To achieve this at least approximately, a lacquer coating can be applied and/or provided between the conductor traces, corresponding to the negative of the conductor trace networks and/or conductor traces. However, since today's lacquer coating methods are not absolutely precise, there may be a slight displacement in the pressure and the location where the pressure fills the gaps.

An improvement is therefore possible, in which two lacquer coatings are carried out or provided. First, a lacquer coating is provided or applied in the gaps, which coating is somewhat narrower and/or smaller than the negative of the conductor trace networks and/or conductor traces and/or than the space between the conductor traces, so that a displacement of the lacquer coating will not cause a coating to be provided on and/or applied to the conductor traces. This first lacquer coating is relatively thick, in particular 10 to 30% and/or 3 to 10 μm less than the conductor traces; for example, with conductor traces that are 35 μm in height, the coating is 30 μm thick, for example.

A second coating is provided and/or applied subsequently and/or to the first, which is thinner than the first, in particular 2 to 20 μm thick. This lacquer coating is somewhat larger and/or wider than the gap between the conductor traces and/or than the negative of the conductor trace networks and/or conductor traces, so that even if a displacement occurs, the conductor traces will be partially covered (to a small extent).

The lacquer coating, or a first such coating, roughly fills in the spaces between the conductor traces, in particular, and the second lacquer coating ensures the smoothest possible transition from conductor trace to lacquer coated space.

In addition to the conductor trace arrays, conductive layers, and/or conductor trace networks and/or sections, which are embodied, in particular, in the manner of an SRS, VRS, and/or FSR sensor, the pressure sensor unit, which is configured in particular as an SRS, VRS, and/or FSR sensor, has in particular a device, in particular elastic, for the targeted transfer and/or distribution of the pressure, and/or a functional polymer. In particular, the functional polymer has at least one conductive surface section, which may be part of the functional polymer, for example, or may be present in the form of a coating. Typically, however, it is formed from a plurality of materials, in particular polymers, that have different properties. The functional polymer has a structural form, in particular, or is embodied as such. It thus can be the structural form, also called the molded body. The structural form or the molded body has, in particular, a discontinuous thickness over its cross-section. In particular, it is spherical, and/or is embodied as a spherical segment, and/or is elastic. The adjustment or the optimization of the functional polymer requires accurate knowledge of the measuring conditions.

For the measurement of blood pressure, the pulse wave, and/or cardiac output and/or changes in cardiac output, this means that the sensor will be used on the skin and that forces in the range of 1-10 N will occur. A high data acquisition rate of at least 1000 values per second should also be possible.

Advantageously, silicone is used as the polymer and/or functional polymer, in particular multiple silicones having different properties.

The requirements in terms of the measuring range and the high data acquisition rate mean that the functional polymer should exert a certain counterforce in order to react to a change in force.

This is made possible in two ways, in particular. The first way involves adjusting the Shore hardness of the silicone, and the second way involves selecting a geometric configuration of the structural form that will enable the required counterforce.

It is also possible for the two properties to be adjusted separately. For this purpose, a structural form is produced, which contains a material that has a higher Shore hardness than at least one other material used in the structural form. This structural form is larger, in particular, than the surface area of the conductor traces, sections, arrays, and/or conductor trace networks, in particular larger than the surface area of the conductor traces, sections, arrays and/or conductor trace networks of an array that is associated with the structural form. Such a structural form, in particular at a point thereon that comes into contact with the conductor traces during use, has a surface area that is formed by a silicone having a different, lower Shore hardness, and in particular is resistance-conductive and/or conductive. In addition to silicone, in particular all polymers, commonly referred to as rubber, that fulfill the mechanical properties described below are suitable.

The functional polymer can also be embodied as having a location-dependent variable Shore hardness. This is accomplished, in particular, by applying the functional polymer, e.g. in layers, and by using a different Shore hardness from layer to layer.

Another possibility for achieving variable Shore hardness involves the use of special UV-curable polymers. These polymers change their Shore hardness when exposed to UV light. Shore hardness can be adjusted dependent upon the exposure time. In this way, for example, different Shore hardnesses can be arranged concentrically either by using masks or by directing UV laser light accordingly. For use, such sensors based on UV-variable polymers must be constructed such that no light reaches the polymer, so that the Shore hardness is maintained.

The shape of the functional polymer, in particular its structural form, is a further adjustment variable. The task of the functional polymer is, in particular, to nestle against the conductor traces when force is applied and to increase the contact surface area dependent upon the force. A roughly hemispherical shape or spherical cap has proven advantageous as a base shape. The outward flattening and the configuration of the central point are adjustment parameters.

In particular, the functional polymer and/or the structural form have a layer of resistance-conductive polymer, in particular assuming the polymers otherwise used are non-conductive.

Resistance-conductive and/or conductive polymer is available in the form of ink, and its conductivity can be adjusted by adding other polymer inks.

Resistance-conductive and/or conductive polymer has, in particular, a specific resistance between 0.2 and 10 k Ohm/cm/mm\hat{ }2.

In general, the conductance between the conductive layers and/or conductor traces is based upon the intrinsic conductivity and the contact conductivity to the conductor traces. In most cases, adjustment of the intrinsic conductivity, and/or the intrinsic conductivity itself, is less important, as the distance between two conductor traces is typically less than 1 mm. In most cases, the adjustment of the contact with the conductor traces, and/or the contact with the conductor traces itself, i.e., the contact resistance, is more important. This contact is dependent, in particular, upon the surface configuration of the functional polymer, in particular its microstructure.

The task of the functional polymer is, in particular, to push the conductive polymer away from the conductor traces and thus to build up a counterforce, which is accomplished, in particular, by using spacers or feet (see below). When the sensor is subjected to a load, the total force on the conductive polymer and thus on the ability to produce contact between the conductor traces is equal to the force on the sensor minus the counterforce of the feet. Thus, the more counterforce the feet can produce, the greater the measuring range that can be achieved. The feet are generally configured such that when the pressure sensor unit is in a resting position, they ensure the spacing of the functional polymer from the conductor traces and/or the conductive layers.

The counterforce should be selected such that the sensor is able to react quickly enough to a change in pressure caused by the variable pressure wave proceeding from the cardiac pulse in the arteries, i.e., enabling a mapping over time, in particular with less than 10% error, in particular based on the duration of a pulse wave and/or in particular of less than 2 ms, more particularly of 1 ms or less, and/or an error in amplitude of less than 10%, in particular of the maximum measurable amplitude and/or the maximum amplitude induced by the pulse wave. For measuring the pulsatile pressure wave in the arteries, a material that has proven advantageous for the spacers, for the functional polymer, for the structural form, and/or for the spherical cap has a Shore-A hardness, in particular according to ASTM D2240 (2015-08) and in particular with a test time of 1 second, of between 85 and 98, and in particular has a Shore-A hardness of between 90 and 98, in particular between 92 and 97, more particularly between 94 and 96, in particular of 95 for arrangement on conductor traces and/or for the electrical connection of conductor traces, and/or has a Shore-A hardness of between 85 and 95, in particular between 88 and 92, more particularly of 90 for arrangement between conductive layers and/or conductor traces (in particular as is described further below as an alternative embodiment of the pressure sensor unit), and/or has a size, in particular a maximum size, of the functional polymer, in particular in cross-section parallel to the surface extension of the conductor traces and/or conductive layers, of between 1 cm×1 cm and 2 cm×2 cm, and/or has a height of between 0.5 and 3 mm, in particular between 1 and 2 mm, and/or has a total surface area of the spacers, in particular in cross-section parallel to the planar extension of the conductor traces and/or conductive layers, of between 3 and 5 mm\hat{ }2, and/or has a number of 3 to 4 feet and/or 1 to 2 feet configured as rings.

For the spacers, for the functional polymer, for the structural form, and/or for the spherical cap, the use of silicone has proven particularly advantageous. Spacers, in particular feet, and spherical cap are configured, in particular, as integral, in particular together with a connecting section for connecting spacers and spherical cap, and the spherical cap, in particular, has a conductive coating.

In particular, the functional polymer has a structural form in the shape of a spherical cap or a spherical segment, the spherical cap or the segment in particular having a maximum diameter of between 2 and 9 mm, in particular between 4 and 6 mm, and/or a height of between 0.5 and 3 mm, in particular between 1 and 2 mm, and in particular is a cap or a segment of a sphere having a diameter of between 8 and 30 mm. In particular, the spherical cap and or the segment has a coating of conductive polymer. The functional polymer of the structural form and/or the spherical cap are made of silicone, in particular. In particular, feet are arranged laterally next to the spherical cap and/or the segment.

Conductor traces, feet, functional polymer, conductive coating, and/or structural form are configured and arranged, in particular, such that in a resting state, a distance of between 0.05 and 0.5 mm, in particular between 0.05 and 0.2 mm, exists between the functional polymer, in particular the conductive coating on the spherical cap, and the conductor traces.

Smaller functional polymers should have a softer polymer or a smaller total surface area of the spacers to enable a desired deformation, but a softer polymer will do a poorer job of following the pulse wave due to its lower rebound elasticity.

This is the basis for the importance and/or the tasks of the individual polymers. The task of the polymer of the structural form and/or the structural form itself, in particular of a resistance-conductive and/or conductive polymer, in particular of a resistance-conductive and/or conductive coating, is first, to guide the unloaded sensor into a defined baseline state, and second, to establish a defined rebound pressure. This rebound pressure is useful for suppressing mechanical shaking, which can in turn produce noise in the measured values.

The task of the functional polymer, in particular the resistance-conductive and/or conductive polymer, in particular a resistance-conductive and/or conductive coating, is to control the contact surface area between the resistance-conductive and/or conductive polymer and the conductor traces. The functional polymer, in particular the resistance-conductive and/or conductive polymer, in particular a resistance-conductive and/or conductive coating, should be characterized, in particular, by a very rapid rebound capability, as one aspect. In other words, the speed of the expansion and release movement should enable it to follow the pulsatile pressure wave. As another aspect, the functional polymer, in particular the resistance-conductive and/or conductive polymer, in particular a resistance-conductive and/or conductive coating, is characterized by the fact that it conforms to the conductor traces to a greater or lesser degree, depending upon the mechanical load. In this respect, the mechanical properties of the functional polymer, in particular of the resistance-conductive and/or conductive polymer, in particular of a resistance-conductive and/or conductive coating, should also be adjusted such that a lasting impression of the conductor traces cannot form in the polymer, even with frequent loading of the sensor up to the maximum pressure range.

The resistance-conductive and/or conductive polymer produces the contact between the individual conductor trace networks. Its properties are, in particular, that actually, i.e. in the idle state of the pressure sensor unit, the polymer does not have effective contact with a conductor trace. This is due, in particular, to the fact that the microstructure of the surface of the polymer, in particular of the resistance-conductive and/or conductive surface, is very uneven. When the polymer comes into contact with a conductor trace, initially only relatively few microstructural protrusions are in contact with the conductor trace, and the electrical resistance between the polymer and the conductor trace is high. As pressure on the polymer increases, the microstructure becomes deformed, and the flattening of the microstructural protrusions causes the actual contact surface area to increase, electrical contact improves, and the electrical resistance between the conductor trace and the polymer becomes low. When pressure is released, the pressure on the flattened microstructural protrusions is also released and the polymer returns to its original shape.

The functional polymer and/or the structural form also have, in particular, at least one spacer and/or foot, which is configured, in particular, to keep the functional polymer, in particular the resistance-conductive and/or conductive polymer, in particular a resistance-conductive and/or conductive coating, spaced far enough from the conductor traces, conductor trace arrays, and/or conductor trace networks to prevent electrical contact when the pressure sensor unit is in an idle or unloaded state. Feet may be configured, for example, as individual protrusions or as concentric structures. They may be adapted in terms of shape and proximity to the functional polymer, in particular to the resistance-conductive and/or conductive polymer, in particular to the resistance-conductive and/or conductive coating, such that the desired counterforce of the sensor is achieved. At the center of the functional polymer, in particular of the resistance-conductive and/or conductive polymer, in particular of the resistance-conductive and/or conductive coating, a protrusion that protrudes beyond the shape of the functional polymer, in particular of the resistance-conductive and/or conductive polymer, in particular of the resistance-conductive and/or conductive coating may be inserted. This protrusion or spacer and/or these feet may have a different Shore hardness or the same Shore hardness as the rest of the functional polymer and/or the rest of the structural form.

Feet can also assume the task of holding the polymer in position, so that ideally, when the sensor is subjected to shear stress, no lateral movement of the polymer over the conductor traces will occur.

It has been found that cavities or depressions in the printed circuit board or in a substrate on which the conductor traces are arranged, in which the spacers and the feet are partially accommodated, or at the locations of the feet, can effectively stabilize the feet or spacers, preventing any lateral movements.

The at least one spacer is glued, in particular, to a substrate on which the conductor traces are arranged and/or to a conductive layer, in particular in cavities or depressions in said substrate.

Additionally, the positions and the shape of the feet should be appropriately chosen. When subjected to a load, few small feet will result in an uneven stress field on the functional polymer. As a result, the functional polymer will not deform concentrically to the applied load.

Optimization can be achieved by selecting an annular and/or concentric foot shape. Said ring has two essential parameters:

The thickness or width of the ring determines how much force the sensor can absorb before it is squashed. The thickness should be selected such that in the targeted measuring range, the most linear relationship possible between the application of pressure and the measured value is produced.

The height of the ring determines the force at which the sensor will begin to supply a signal. In order for an optimal and well-resolved measurement to be performed, the height should be selected such that measurement begins only after a reasonable application of pressure.

The two parameters may influence one another. The taller the ring, the thicker it will become under deformation when the sensor is loaded. A tall ring will also lead to an increase in force absorption.

In particular, if the remaining functional polymer and/or the remaining structural form is not resistance-conductive or conductive, a further resistance-conductive and/or conductive polymer is applied to the functional polymer and/or the structural form, and/or, if the remaining functional polymer and/or the remaining structural form is not resistance-conductive or conductive, the functional polymer and/or the structural form has a resistance-conductive and/or conductive polymer. The size of the functional polymer is dependent, in particular, on the surface area of the conductor trace networks.

An alternative pressure sensor unit contains a plurality of sensitively active surface areas or volumes, one above the other and/or into one another. This allows multiple measuring ranges to be created, for example, which can be used simultaneously at the same measuring point.

The configuration of polymer pressure sensors described thus far has been based on a conductive polymer being pressed against two separate networks of metallic conductors. Depending upon the applied pressure, the electrical contact between the metallic conductor traces and the polymer changes. The polymer produces a resistive contact between the two conductor trace networks. Here, the resistance is equal to the sum of the contact resistances between the polymer and the two conductor trace networks and the resistance of the polymer, per se.

An alternative pressure sensor unit includes two surface areas or layers of conductive material, in particular polymer. These surface areas or layers are arranged one above the other and are separated by an additional layer or surface area of a non-conductive polymer or non-conductive lacquer. The two surface areas or layers of the conductive polymer are electrically contacted, and the resistance and/or conductance between the two surface areas, in particular, is measured.

Advantageously, the Shore hardness of the conductive and the non-conductive polymers is selected such that in the desired measuring range, the polymer will deform when subjected to a load.

The shape of the surface areas or layers need not be flat, and can instead be adapted to the measuring system or to the measuring task. For example, a configuration is possible in which polymer layers are layered in the form of a finger pad, allowing vital data to be recorded at the radial artery. The sensitive surface area is advantageously on the surface of this finger pad shape and is therefore curved.

The shape of the non-conductive surface area or layer, in particular, determines the potential measurable measuring range.

In the simplest case, at least one hole, in particular holes, is introduced and/or provided in the non-conductive layer, for example, non-conductive polymer or non-conductive lacquer, forming at least one air-filled cavity. When pressure is applied to the sensor, the non-conductive layer or surface area deforms, or the three surface areas or layers deform, and the upper and lower surface area or layer of conductive polymer contact one another. A resistive electrical contact is thereby produced. Depending on the pressure load applied, the contact surface area and the pressure of the surface areas against one another become greater, and contact occurs in more and more cavities and/or over larger surface areas. This reduces the electrical resistance or increases the conductivity between the surface areas of the electrically conductive polymers.

An improved configuration of the cavities between the conductive polymers can be achieved by increasing the maximum contact surface area per cavity or hole or overall. For this purpose, at least one hole is introduced into the non-conductive surface area or layer, as usual, and conductive material, for example, in particular polymer, is additionally placed in the at least one hole or in the holes in order to produce extensions of the conductive layer in the holes. Advantageously, one or both conductive layers, in particular at least one extension thereof, protrude(s) into at least one hole, in particular in such a way that in an idle state, no contact is made between the conductive layers in the at least one hole or in the holes. The arrangement or extensions may be hemispherical and/or spherical cap-shaped, in particular, and/or in a negative shape thereto, and/or may have a first shape on the lower conductive surface with a counterpart complementary to the first shape and/or a roughly complementary counterpart on the upper surface. A hollow hemisphere thus meshes inside a solid hemisphere, for example. Meshing spherical caps and negative shapes thereto, having different or equal radii, can also be used for the two conductive surface areas.

The range of force of such an assembly can be adjusted, for example, by altering the number of cavities and the shape of the conductive polymer arrangement in the cavities. The size and shape of a cavity is another parameter, along with the number of cavities per unit of surface area. Further adjustment parameters include the thickness of the surface areas and their hardness, in particular that of the non-conductive surface.

In addition, 3D printing enables additional options for the mechanical adaptation of the sensors for adjusting a measuring range.

In such a configuration of the pressure sensor unit, having a non-conductive layer between two conductive layers, in an idle state, the insulating polymer and/or the insulating lacquer has a thickness, in particular, of between 0.5 and 2 mm, and/or, in an idle state, the conductive layers, in particular the extensions thereof, are spaced apart from one another by between 0.05 and 0.5 mm, in particular between 0.05 and 0.2 mm. In particular, the conductive layers and/or the insulating polymer and/or the insulating lacquer each have a surface extension of between 0.5 cm\hat{ }2 and 9 cm\hat{ }2, in particular between 1 cm\hat{ }2 and 5 cm\hat{ }2. The insulating polymer and/or the insulating lacquer, in particular, have 3 to 15 holes and/or holes having a total surface area of 50 and 200 mm\hat{ }2, in particular per 1 cm\hat{ }2 to 5 cm\hat{ }2 total surface area of the insulating polymer or lacquer, and/or the holes each have a surface area of 10 to 40 mm\hat{ }2.

For the hardness of the non-conductive layer, a Shore-A hardness, in particular according to ASTM D2240 (2015-08) and in particular with a testing time of 1 second, of between 85 and 98, between 85 and 95, in particular between 88 and 92, in particular of 90, has proven advantageous. It is made of silicone, in particular.

The above-described cavities or holes contain air, most of which cannot escape, and thus, when subjected to pressure, the pressure in these cavities increases. A change in pressure application with a low (base) pressurization produces a different deformation from the same change in pressure application with a higher (base) pressurization, due to the different air pressure in the sensor.

This phenomenon can be remedied by creating or incorporating an opening to allow pressure equalization, for example connecting the cavity to the environment, or this phenomenon may be used as a dynamic adjustment parameter for the sensor during use.

When an opening is created and/or located between the cavities and a pump, the pump can be used to generate pressure in these cavities, thereby altering the deformability of the cavities. The more pressure there is in the cavities, the more difficult it is to deform the cavities, meaning that greater pressure application is required for deformation. The measuring range is can be adjusted by adjusting the pressure in the cavities.

Thus far, only the configuration of a sensor having a measuring range or having a dynamically adjustable measuring range has been presented. It is likewise possible for multiple independent measuring ranges to be used. For this purpose, additional areas of non-conductive and conductive polymer are applied and/or arranged alternatingly. Each additional area of conductive material increases the range of measurement by one.

If two measuring ranges will be used, then three areas of conductive polymer, separated by two areas of non-conductive polymer, are required. For different measuring ranges, the structural parameters of the cavities between the first two conductive areas are and/or should be set differently from the structural parameters of the cavities between the second and third conductive areas.

Another possible configuration of multiple measuring ranges within a sensor involves using only two areas of conductive polymer. However, one or both areas is/are constructed from strips. These strips are in turn separated from one another by non-conductive polymer. Each strip is associated with a measuring range, and with two measuring ranges, the association with an area is alternating, in particular. The strips that form a set are electroconductively connected outside of the sensor. The parameters of the cavities are then adapted and/or are designed as different for each group of strips, i.e., for the corresponding measuring ranges.

If an extremely high number of measuring ranges is required, these two strategies may be combined in order to increase the measuring ranges.

When an arrangement is used in which two areas composed of strips that are rotated in relation to one another and are arranged one above the other, so that the strips of one area are perpendicular to the strips of the other area, the location of the load can be determined. It is also possible for multiple areas composed of strips that are not necessarily aligned perpendicular to one another to be used. Each strip is electrically contacted individually and the resistance between any pairs of strips, consisting of one strip on each area, can be determined. The point where the highest load is found is the point of intersection of the pair having the lowest resistance.

In addition to actual sensor production, 3D printing also offers the option of integrating one sensor or multiple sensors in the housing or fastening elements of a wearable. With the aid of 3D printing, the housing or the fastening elements of the wearable can be adapted optimally to the measuring task and equipped with functional features. 3D printing allows the integration of an applied pressure system, which operates, e.g., on the basis of 3D-printed pneumatic components, or the integration of additional sensors, such as plethysmography-based sensors that use special light-conducting 3D-printable materials or air cuffs with pneumatic air pressure sensors to measure blood pressure by the Riva Rocci method. Thus, the housing having the fastening elements is a characteristic, essential development for an assembly for collecting vital data.

Determining the cardiac output of the cardiovascular system as a parameter is possible, in particular, if other parameters, such as blood pressure and cardiac pulse, are first determined. Therefore, in the following, all parameters that can be measured using an assembly according to the invention will be defined, with cardiac output being defined last.

The blood pressure is detectable at various points on a human or animal body, and sometimes is even visible to the human eye.

In particular, in humans the arterial pulse at the radial artery is visible with the naked eye.

Thus, the blood pressure from the heart is not only palpable but also visible, even in the peripheral parts of the human or animal.

This pressure is particularly perceptible in the wrist. Here, the radial artery (see FIG. 1, (L)) extends beneath the skin, but is close enough to the surface that the attenuating properties of the tissue (see FIG. 1, (C)) are partially mitigated. When viewed closely, pulsating tissue is usually visible.

This pulsation is produced by the change in pressure over time with each individual heartbeat.

During the expulsion period of the individual heart rhythms, the pulse wave (see FIG. 1, (D)) is also generated. This can be registered peripherally as the first reaction of a successful heartbeat.

However, a portion of the blood volume is not delivered immediately to the peripheral regions. The windkessel receives a portion of the blood volume and discharges this portion within a RR interval until it is emptied completely, measurable as diastole.

This occurs to compensate for pressure peaks and to allow the system to better adapt to respective load conditions.

When it arrives in the peripheral regions, the pressure pulse or the current pulse can be registered.

For more than 100 years, the Riva Rocci method has been used to measure blood pressure non-invasively.

This method involves squeezing and occluding the artery with the aid of an inflatable air bag. This uniform external application of pressure on the artery is then released in a controlled and measurable manner by opening the air bag.

The arterial blood pressure then causes the blood to pass through the first opening slits in the occlusion to the peripheral region, and thus spread again within the open artery, producing a tapping sound. The Riva Rocci/Korotkow method uses this tapping to measure blood pressure.

When a sound of the re-established blood flow is detected, this signifies the systole (See FIG. 1, (A)).

When the tapping ceases, the pressure in the arterial vessel is normalized and the pressure of the diastole (See FIG. 1, (B)) can be measured.

Closer consideration of the measuring instruments used in the Riva Rocci/Korotkow method reveals the following: When an analog pressure gauge is used to determine the pressure in the air bag, pulsation of the gauge needle and thus of the pressure can be observed. This is observed when the air bag is pressurized with a pressure that is between diastole and systole. In that case, the air bag is inflated with air in which the fluctuation is at its maximum.

The values between which the needle fluctuates would correspond to the values for diastole and systole. However, since the air bag attenuates the change in pressure, and since the measuring gauge does not have the necessary temporal resolution, the values for diastole and systole cannot be determined in this way.

The assembly according to the invention makes use of this fact by employing sensors that have the appropriate temporal resolution and that have the lowest possible attenuation.

The invention uses the incoming pressure/pressure propagation direction to the surface of the human skin (see FIG. 1, (N)), in this case, for example, at the radial artery.

The pulsatile pressure proceeding from the arterial wall (see FIG. 1, (E)) propagates across the tissue (see FIG. 1, (C)) to the surface of the skin (see FIG. 1, (O)). There, the pulsatile pressure/blood pressure, already attenuated by the tissue, can be registered by means of a sensory and processing unit (see FIGS. 1, (K) and (H)).

Directly measurable pulse pressure has been a proven tool used by medical professionals for centuries, not just in the field of emergency medicine. The nature of the pulse pressure propagation, FIG. 1, (N), and the rate of the pulse, FIG. 1, (M), can allow inferences to be drawn regarding the overall health of a patient based upon the palpated pulse.

In the invention, the registrable pressure pulse is used directly as a basis for blood pressure measurement.

The pulsatile pressure pulse deforms the artery, FIG. 1, (E), steadily during the cycle of the beating heart, FIG. 1, (G). On its way to the peripheral regions, the pressure pulse is altered by branching, by the conditions of the vessels, and by the external and internal load.

Thus, the blood pressure measurement is a value that adapts constantly to the respective conditions of the body.

The functioning of the heart is controlled by the sinus node, which determines the rhythm with which the heart contracts, causing the blood to be ejected first into the windkessel and then into the arteries. The sinus node uses electrical signals to stimulate the muscles of the heart.

A cardiac pulse is a full cycle of the ejection of blood into the arteries and the return of blood from the veins.

In the conventional method for measuring the cardiac pulse using an electrocardiogram (ECG), the electrical signals of the sinus node are recorded.

However, this recording of these electrical signals, although used worldwide, is inadequate for measuring the cardiac pulse, because the electrical signal is merely the instruction to the muscles of the heart to perform an action; the actual action of the heart cannot be detected in this way.

In cases of illness, the electrical signal of the sinus node may not cause an ejection of blood. One example of this is atrial flutter and atrial fibrillation. With these diseases, contraction in the atria of up to 340 (atrial flutter) or up to 600 (atrial fibrillation) contractions per minute is triggered by electrical signals. But in most cases, the heart valves to the ventricles open irregularly at a rate of 100-160 openings per minute. This condition is called absolute arrhythmia.

The result is an ejection of blood at a rate of only 100-160 per minute.

Of course, such conditions produce detectable patterns in the ECG signal that can be recognized by those skilled in the art. However, this is dependent upon the experience and training of those skilled in the art, and above all on their knowledge of all possible diseases.

The assembly according to the invention detects measured values that map the pulsatile pressure wave. The pulsatile pressure wave in the arteries is generated only when blood is ejected from the heart. Thus, by analyzing the measured values that map the pulsatile pressure wave, a clear measurement of the cardiac pulse can be obtained.

The pulse wave variability, also called heart rate variability, indicates the variability of the cardiac pulse. High variability is a sign of a healthy heart.

The cardiac pulse adapts autonomously to the requirements of the organism and is therefore subject to constant change. If the patient being examined is under elevated stress, for example, the result may be a uniform cardiac pulse.

By recording a measurement curve that maps the pulsatile pressure wave, the assembly according to the invention enables the time intervals between each individual cardiac pulse to be determined. These intervals are called RR intervals.

Pulse wave variability can be expressed as the standard deviation from the mean of the RR intervals, for example.

The pulse wave transit time and pulse wave velocity are two closely related parameters of the cardiovascular system.

The pulse wave transit time indicates the time required for the pulse wave to travel a certain distance, and the pulse wave velocity combines the pulse wave transit time with the distance traveled. Therefore, if the distance traveled is known, the two parameters can be converted to one another.

Moreover, the pulse wave velocity should not be confused with the velocity of the blood in the arteries, which is much slower.

Proceeding from a cardiac pulse, a pressure pulse begins to move within the arteries, deforming the arterial walls. Conversely, this means that the pressure pulse is able to advance only as rapidly as the arterial walls can deform. This deformability is the elasticity of the arteries.

Thus, the elasticity of the arteries can be determined by measuring the pulse wave velocity. To determine arterial elasticity, a person skilled in the art may use, inter alia, the Moens-Korteweg formula and/or the Bramwell & Hill formula, which indicates the dependence of the pulse wave velocity on the elasticity, the arterial wall thickness, the arterial diameter, and the density of the blood. The elasticity value is regulated by the cardiovascular system.

In cases of illness, elasticity may be reduced excessively, e.g., by arteriosclerosis. Elasticity is therefore a parameter that may indicate an impending heart attack, for example.

Moreover, studies have shown that, at least over short periods of time, pulse wave velocity can also be used to monitor blood pressure.

For measuring pulse wave velocity using the assembly of the invention, there are two approaches, in particular.

Firstly, the pulsatile pressure wave can be measured by multiple sensors at various points on the body. The pulse wave transit time can thus be determined from the relative offset of the measurement curves, and therefore, assuming the distance between the sensors is known, the pulse wave velocity is also known.

Since the assembly of the invention has a high data acquisition rate, the sensors can also be positioned close to one another. This allows the pulse wave velocity to be measured in a location-specific manner on the body.

Secondly, the pulsatile pressure wave of the cardiac pulse is also reflected. Due to the high data acquisition rate of the assembly of the invention, the reflected wave is discernible in the measurement curves. The pulse wave transit time can also be calculated from the distance between the initial wave and the reflected wave.

The cardiac output indicates how many liters of blood the heart ejects in one minute and is available for supply to the organism. Thus, this parameter or the change in this parameter is the variable that is used to assess the performance of the cardiovascular system.

A value that is lower than normal is an indication of a heart disease, e.g., a valvular heart disease, or is an indication of hypothyroidism.

A value that is higher than normal may be due to a variety of disorders, e.g., fever, anemia, or circulatory disorders of the organs.

Cardiac output is also an indicator of oxygen supply to the body. For athletes, therefore, a high cardiac output is desirable (as long as this is not disease-related) and can serve as a measure for specific, performance-oriented training measures.

Another area of application that is based on the connection to the supply of oxygen involves the monitoring of patients during operations. With many surgeries, it is already standard procedure to measure cardiac output continuously, but this is done invasively using a variety of methods. One method that is frequently used involves using a catheter to inject a cold fluid into a ventricle of the heart. Using temperature sensors in the arteries downstream from the heart, the degree of heating can be determined; this heating is directly related to cardiac output.

In sum, the methods for determining cardiac output are either highly imprecise or require invasive procedures in the body that are justifiable only in exceptional situations.

An assembly according to the invention offers an alternative to invasive methods that requires no invasive procedures in the body and thus no dangerous operations, while at the same time enabling the most accurate measurement possible of cardiac output or changes therein. Its area of application therefore lies between the medically highly precise monitoring situations that are essential during complex and extended surgeries and the highly imprecise, already non-invasive methods. Therefore, during surgeries that do not require the most accurate reading possible, an invasive method can be dispensed with and an assembly according to the invention can be used in its place. Since an assembly according to the invention can be fitted quickly and easily onto a patient, monitoring is possible during surgeries in which no cardiac output monitoring has heretofore been used.

Furthermore, specifically in the medical field, new possibilities are opened up that will enable continuous long-term monitoring, and in the event of deterioration, the triggering of an alarm.

The value for the current cardiac output can be determined from cardiac pulse to cardiac pulse. The parameters of the cardiac pulse rate or the RR interval, the pulsatile pressure wave measurement curve, arterial elasticity, and for diastolic blood pressure the diameter of the aortic arch are determined.

Only the absolute diameter or radius of the aortic arch with the diastolic blood pressure cannot be determined using an assembly according to the invention, and must be ascertained using external instruments, e.g., ultrasound. However, for continuous long-term monitoring, the changes are more important than the absolute value. An assembly according to the invention can measure cardiac output at least approximately relative to the diameter and/or the cross-sectional area of the aortic arch with the diastolic blood pressure, and if desired, this relative value can be converted to an absolute value by an external measurement of the diameter of the aortic arch with diastolic blood pressure.

The pulse wave travels from the heart into the arteries, during which time the amplitude of the wave decreases and the individual pulses become longer temporally. However, the output pressure wave must pass through all the arteries, and the overall pressure as an integration over time, in particular of one pulse wave, one RR interval, and/or between two systolic pressures or two diastolic pressures, at a certain distance from the heart is approximately equal to the ejection pressure associated with cardiac output and elasticity. Elasticity, in turn, can likewise be at least approximated, and can be used for further refinement as a correction of the at least relative cardiac output.

Thus, the ejection volume of a cardiac pulse, even without additional correction for elasticity, can be approximately determined as follows: The current pulse pressure P(t) (pulse pressure is the pressure difference between the diastolic blood pressure value and the current measured value) in an artery deforms the artery, and the radius R is determined by the elasticity E:

R(t)=R₀+E*P(t)

Here, R₀ is the (unknown and externally measured) radius of the artery. Thus, the current radius of the artery changes during a cardiac pulse with the pressure over time.

To calculate a volume, the length L of the deformation of the artery during a cardiac pulse must be determined. For this purpose, the length over time of a cardiac pulse, the RR interval T, and the pulse wave velocity v are correlated:

L=v*T

Since the current time t (starting time is the beginning of the cardiac pulse, with t=0) in the pulse at the current position of the pressure wave in the artery l(t) can be correlated to the pulse wave velocity:

l(t)=v*t

The current radius can also be expressed as a function of l. R′(l)=R(l/v)

The volume is obtained by integrating all measured values in a cardiac pulse under the known formula for determining cross-sectional area from the radius (area=πR²):

V=∫π(R′(l))²dl=π∫(R(l/v))²dl=π∫(R(t))²dt

π∫(R₀+E*P(t))²vdt

For a relative measurement, R₀ is set to zero, and if an external measurement is taken, R₀ can be transmitted as a parameter to the assembly of the invention.

A pressure sensor unit as described above can be placed on the skin; FIGS. 1, (K) and (O).

To achieve evenly applied pressure, a wristband, FIG. 1, (I), may be used around the wrist as an aid and as a commercially available product. In this way, an even pressure, FIG. 1, (J), can be exerted on the radial artery.

The outgoing pressure transmitted from the radial artery to the surface of the skin can be registered by even a simple measuring configuration composed of a pressure sensor unit between the wristband and the surface of the skin.

The dynamic pressure pulse of the actual ejection from the heart can thus be made visible to anyone by means of an analysis and imaging unit. The assembly according to the invention is therefore of inestimable value to patients who are invasively connected to a monitor, for example, in an intensive care unit, since measurement does not have to be performed invasively, as is currently customary.

The measurement of blood pressure can be influenced greatly by the surrounding tissue. Allowances should therefore be made for this in special cases.

In people who have a high percentage of body fat or who are moderately stocky, the preferred location for measuring blood pressure and cardiac pulse on the wrist may result in increased attenuation of the measurement.

In such cases, the upper back of the foot is an advantageous measurement site, since usually, little fat is stored there. Measurement sites such as the dorsalis pedis, anterior tibial, posterior tibial, first dorsal metatarsal, deep plantar, and arcuate arteries are suitable measurement sites at the back of the foot. In the area above the ankle, the total volume of the inflowing bloodstream changes the diameter of the lower limb. By measuring the variable diameter or the variable pressure on the impinged measuring surface area using at least one pressure sensor unit, the blood pressure and cardiac pulse can also be detected here.

The invention can also be used to a limited extent in people suffering from conditions, for example, that involve substantial accumulations of fluid in the legs and particularly in the feet.

The counterpressure and/or applied pressure that is required may also be generated, for example, using a finger (see FIG. 1, (J)) on the other free hand.

For this purpose, a specially constructed surface that is clearly identified for the user on the wristband or system can preferably be created for the invention. This surface is located above the at least one pressure sensor unit and thus on the wristband, for example. Directly below the pressure sensor unit is the radial artery, for example.

For blood pressure measurement, the user can place one finger on the specified surface of the wristband, for example. A gently and steadily rising increase in pressure on the wristband, produced by a building pressure of the finger on the marked surface area, is registered and stored by the system, in particular with the pressure sensor unit, at a rate of at least 1000 measurements per second.

When gentle pressure is applied to the radial artery, the user will feel an increasingly perceptible pulsation. This increase in the perceptible pulse is due not to an increase in blood pressure but to the compaction and distribution of the surrounding tissue between the surface of the skin and the tissue above the radial artery.

Like a squeezed-out sponge, the compressed and displaced tissue transmits the pulsatile wave nearly 1 to 1 from the arterial wall to the pressure sensor unit. The attenuation of the tissue, FIG. 1, (C), is reduced by the counterpressure and/or applied pressure applied by the finger or an actuator, FIG. 1, (J), from the outside and thus above the skin, FIG. 1, (O).

To allow the systolic blood pressure to be measured, the counterpressure on the artery or pulse wave (see FIG. 1, (D)) should be increased beyond the point of attenuation.

The systole can be measured at the moment when, as the counterpressure and/or applied pressure continues to increase, the pulse wave is not able to generate a maximum increase beyond the maximum measured pressure.

People can easily test the invention on themselves. Before the radial artery is occluded completely by the increasing pressure applied to the radial artery, there is a pressure range in which, despite an increase in the counterpressure, no further perceptible increase in the pulse pressure occurs.

This point can be measured by known devices and represents the systole of the blood pressure.

The diastole is determined from the minima of the pulsatile pressure wave. As the counterpressure on the artery increases, the distance between the minima and maxima of the pulsatile pressure wave initially increases, with the maximum measured pressure also increasing. Beyond a certain counterpressure, the maximum measured pressure does not increase any further. This is the counterpressure that corresponds to the diastolic blood pressure.

As the counterpressure increases further, the minima continue to increase, while the maxima of the pulsatile pressure wave remain constant.

A one-time measurement can therefore be taken by slowly increasing the counterpressure on the artery until no further increase in the maxima of the pulsatile pressure wave are observed.

Continuous measurement is initially carried out in precisely this way, in particular. However, at the point at which no increase in the maxima is observed, a pause occurs, in particular. If the systolic blood pressure then changes, this is detected by a change in the maxima of the pulsatile pressure wave. In that case, the counterpressure must be readjusted to determine the new blood pressure value, more particularly the counterpressure should be increased starting from a value below the systolic blood pressure until no further increase in the maxima is observed.

If only the diastolic blood pressure increases, the distance between the minima and the maxima in the pulsatile pressure wave will change. In that case, the counterpressure should be readjusted, more particularly, it should be increased starting from a value below the systolic blood pressure until no further increase in the maxima is observed.

Only the detection of the unlikely case of a dropping diastolic blood pressure with a constant systolic blood pressure requires a periodic readjustment of the counterpressure, in particular a repeated increase in the counterpressure and/or applied pressure, more particularly an increase in said pressure starting from a value below the systolic blood pressure until no further increase in the maxima is observed.

Measurement of the pulse wave transit time and the pulse wave velocity requires measurement of the pulsatile pressure wave at various points simultaneously. For this purpose, pressure sensor units are attached to at least two suitable measurement sites and are pressed against the skin manually or by means of an actuator with the counterpressure and/or applied pressure, more particularly are loaded with the optimum measuring pressure for continuous measurement, and/or with a counterpressure and/or applied pressure within the range that extends between the pressure of the pulse wave in the diastole or the determined diastolic blood pressure and the pressure of the pulse wave in the systole or the determined systolic blood pressure, and/or up to 1.5 times, in particular 1.3 times the pressure of the pulse wave in the systole and/or the systolic blood pressure and/or from 60 to 120 mmHg, in particular from 60 to 90% of the systolic pressure of the pulse wave in the systole, in particular at the site of measurement and/or the lowest possible pressure that is still sufficient to map the pulsatile pressure curve using the at least one pressure sensor unit. The distance between the pressure sensor units, if the pressure sensor units are separated, must then be measured and transmitted to the assembly of the invention. In the case of a sensor array, these distances of the assembly of the invention are typically known and/or constant. The pulse wave transit time and the pulse wave velocity can then be calculated automatically.

For blood pressure measurement, only one pressure sensor unit is required, however the use of multiple pressure sensor units is advantageous.

Taking multiple measurements of the current blood pressure at each measurement site enables the progression of the blood pressure between diastole and systole in each individual pulse to be plotted. This is particularly advantageous for assessing the cardiovascular system. For example, the reflected wave can be detected. If the reflected wave is increased over the initial pulse wave or pressure wave, for example, this is an indication of vascular stiffness. Examining the reflected wave is one possible option for determining the elasticity of the arteries.

However, blood pressure can also be measured with fewer than 1000 measurements per second.

The invention also offers the solution of generating the counterpressure and/or applied pressure by means of an actuator. The advantage of this is a uniform increase of the counterpressure and/or applied pressure acting on the artery, in particular.

Another advantage of using an actuator is a timely termination of measurement, or a limitation of the counterpressure and/or applied pressure, in particular on the artery, before or so that the counterpressure and/or the applied pressure occludes the artery.

This also enables the blood pressure to be measured automatically, for example during the night, and gently.

The actuator can address the problem of generating counterpressure electrically, pneumatically, hydraulically or manually by way of muscle contractions, for example. The application also allows a combination of all of these solutions with one another and opposite one another.

An actuator comprises, in particular, an air bag and a pump for pressurizing the air bag. An extremity is encompassed, in particular, by an elastic or inelastic encompassing device, for example a band, and between the band and the skin or on the inside of the band an air bag is located between the pressure sensor unit and the encompassing device. To generate the applied pressure, the air bag is pressurized, in particular, especially by pumping in gas, in particular air.

A hydraulic or pneumatic actuator has, in particular, one or more of the following components: tubes, lines, check valve, pump, release valve, shut-off valve, pressure relief valve, buffer volume, and/or actuators.

A system, the use, or the method may be expanded by two additional types of measuring techniques. Firstly, measuring techniques may be applied at the site of the pressure sensor unit. Secondly, measuring techniques that record location-independent values may be integrated.

Measuring techniques that can be used at the site of the pressure sensor unit are: plethysmography, ECG, pneumatic pressure measurement, and/or sound detection.

Measuring techniques that can be integrated are: acceleration sensors, gyroscopes, and/or the detection of environmental parameters.

The system or method or use may also be configured such that the applied pressure is also designed to completely occlude the artery. This allows blood pressure to be measured according to the conventional method, the Riva Rocci method, by means of sound detection.

Measurement according to the Riva Rocci method is achieved, for example, in that in addition to the actuator either a sensor for detecting sounds, e.g., a microphone, a sensor, for example a pressure sensor unit according to the invention, for measuring pressure in a pneumatic applied pressure system, for example an air bag, and/or a sensor for plethysmography is used and/or included.

In the Riva Rocci method, the arteries of the arm are squeezed to the point of occlusion and then slowly released again. When pressure on the arm is released, the arteries begin to open from a certain load level; this is the systolic value of the blood pressure. When the arteries are fully opened, the blood begins to flow normally again. The maximum applied pressure at which normal blood flow is still possible is the diastolic value of the blood pressure. This is measured by examining the sounds produced by the flowing blood. When the arteries are occluded, no noise is produced. When the arteries are partially open, swishing and tapping sounds are produced. With normal blood flow no further sounds can be heard. With today's automated systems, a method is also used which analyzes the pressure in the arm cuff. When squeezing pressure is applied to the arm, the pressure in the cuff is stable over time. When the arteries are partially open, sharp fluctuations are observed. When the arteries are fully open, no measurable fluctuations or very slight measurable fluctuations in the pressure measurement curve are observed.

With a combined sensor, it no longer necessary for squeezing pressure to be applied to the entire arm. If a wristband is used, for example, only the radial artery needs to be occluded, and measurement can be confined to a smaller area. If a microphone is incorporated into the combined sensor, the sounds are analyzed as with the conventional method. The pressure of the applied pressure system can also be used for measurement. The blood pressure values obtained in this manner correspond in terms of accuracy to the values that can be measured using a conventional blood pressure cuff.

However, if a plethysmographic sensor is used, greater accuracy can be achieved. Plethysmography measures the cardiac pulse by radiating light into the tissue and analyzing the intensity of the reflected light. The intensity of the light varies depending upon the filling of blood in the arteries, and since the blood fill varies within a cardiac pulse, the cardiac pulse can be measured. The plethysmographic sensor, in particular, is positioned farther away from the heart than the pressure sensor unit.

If a wristband is used, the plethysmography unit is positioned closer to the hand than the point at which the radial artery is occluded by the applied pressure system. When the radial artery is occluded, no fluctuation in light intensity can be detected. As the artery opens, measurable changes in the light intensity occur, and the artery is pressurized with the systolic pressure. As opening of the artery progresses, the fluctuations in the intensity increase. When the load on the artery is lower than the diastolic value, the fluctuation in the intensity will no longer increase. This method is more accurate than the conventional Riva Rocci method because the actual blood flow is examined and not an indirect sound or a pressure fluctuation within the applied pressure system. Therefore, such a combination of sensors is suitable especially for applications that involve significant movements.

To measure the ECG wave, the pressure sensor unit is equipped with an electrode that is pressed against the skin. In order for a signal to be detected, a second electrode is applied to the surface of the measuring system. The user can then measure his ECG wave by touching this external contact with the other hand, thereby closing the so-called Cabrera circle. The voltage changes between the two contacts are the ECG wave.

The ECG wave is used to examine the heart rate by determining the times of the individual pulses. The times of the pulses correspond to actions of the heart. In this way, the time at which blood is ejected from the heart can be determined. The pressure sensor unit determines the point in time at which the pressure wave generated by the ejection of blood reaches the measurement site. The time difference between these points in time is the pulse wave transit time, or (if the distance between heart and measurement site is known) the pulse wave velocity can be calculated.

On the surface with which the pressure sensor unit rests against the skin, no additional functional feature is housed, and therefore, this surface can be coated with an electrically conductive and flexible material. This may be one of the two electrodes. In particular, a system according to the invention has an encompassing device, in particular a band, and at least one pressure sensor unit, between which an actuator, in particular an air bag, is arranged, along with a pump, in particular, for pressurizing the air bag.

The encompassing device may also be in the form of a shoe, in particular with a closure system. The system may also include a shoe with a closure system, having an actuator and at least one pressure sensor unit in the shoe.

The actuator for generating the counterpressure and/or the applied pressure may be, for example, a conventional electric vibration motor produced in an SMD design. However, any other type of actuator may also meet the requirements for establishing the counterpressure. This is known to those skilled in the art.

Nevertheless, actuators also have the disadvantage that they consume electric power. This must be accounted for in the respective application.

Manual and actuator-controlled closure systems, for example in a shoe or in cuffs, are currently produced by Boa Technology. In 2016, market leaders such as Nike (HyperAdapt 1.0), Puma (Ignite Disc), Reebok with “The Pump technology”, and the French company Digitsole (Smartshoe 002) also installed actuator-controlled closure systems in a product for the first time. Thus, a “shoe” with simple preconditions that can be automated and integrated for continuous pulse and blood pressure measurement already exists.

It is also possible, however, for the required counterpressure and/or applied pressure to be generated using the muscles of the forearm by means of muscle contractions, e.g., by opening and closing the hand. In this way, measurement can be performed by a person even without the use of a finger on the other hand, or without the help of a third party or even of another actuator.

A further advantageous invention involves the use of a convex structure on which a sensor array is located. FIG. 3 shows a convex structure denoted by the letter Q, as a substrate for supporting the system or sensor array.

In most cases, the arterial system is protected and secured inside the body. Often, only the veins are clearly discernible in the extremities. Arterial circulation lies deeper within the tissue. Only at a few points on the body can arteries be felt in a pronounced way through the pulsatile wave. The convex structure, FIG. 3, (Q), is an inventive novelty that allows measurements to be performed even on arteries that are not visible. The convex structure conforms in nearly an exact fit with the concave forms of the body, such as the radial artery. When the convex structure is positioned against the concave shape that forms above the radial artery and faces the surface of the skin, an optimal pulse wave can be registered and stored. The same also applies, for example, to the foot.

To enable generation of a uniform surface pressure on the artery of the foot or the forearm/wrist, it is advantageous to incorporate foam or foam-like material between the flexible sensor unit and the applied pressure component, which is a convex structure, for example. The foam serves as a resonator, similar to the tissue above the bone beneath the finger pad.

The functional Shore hardness of the applied pressure component can be made consistent with the tissue of a finger pad.

The object of correctly positioning the convex structure, which is the size of half a pea, for example, can be attained with the aid of sliding glides on a wristband, for example a watch band, and also with the aid of loops. Even more accurate positioning can be achieved by using multiple sensors; see below.

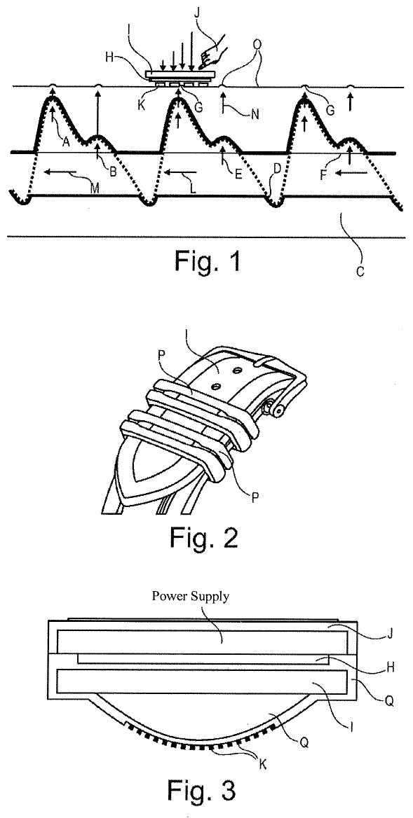

A conventional watch band has at least one loop, FIG. 2, (P), for the overhanging perforated band to enable optimal adjustment of the applied pressure of the watch against the wrist. Without a loop, the overhanging perforated band, which would not be inserted into the loop, (e.g., a leather wristband), would turn away from the shape of the wrist, and stick out. The measuring unit is attached in particular as follows:

The measuring unit or system is slid onto the wristband through the opening, FIG. 3, (I), like a loop(s); see FIG. 2, (P).

Attachment of the measuring unit, having dimensions of approximately 10×20×8 mm, for example, is thus ensured.

The processing and wireless unit and the power supply are located above the wristband, for example; see FIG. 3, (H).

The convex structure with the pressure sensor units is located below the wristband. See FIG. 3, (Q).

With this structural embodiment, the measuring unit or the system comprising the convex structure can measure blood pressure with any wristband already in use.

The inventive novelty thus also consists in the variable usability of existing wristbands for watches, jewelry, or smart devices.

The loop opening of the measuring unit can be made wide enough not only for the convex structure to be moved on the wristband around the wrist, but also for the convex structure along with the pressure sensor unit to be moved closer to or farther away from the hand with the loop, or within the loop.

Thus, the invention can be positioned easily, for example on the surface of the skin, and can serve as a mobile solution for measuring the various parameters of the cardiovascular system.

Advantageously, the measuring unit is a part of the wristband, or the loop, in which case with such an assembly, the wristband and thus the measuring unit is already at a suitable location for blood pressure measurement, and at the same time, pressure can be exerted on the sensor by adjusting the perforated band.

To optimize better positioning of the invention, the measured value can be transmitted to a mobile smart device, for example.

With multiple pressure sensor units in a tiled arrangement within and/or on the convex structure (see FIG. 1, (K), and also FIG. 3, (K)), the optimum location and/or pressure sensor unit of a sensor array for registering the physical pulse wave can be determined. A display for displaying displacement instructions, for example on a smart device, also solves the problem of correct positioning by means of directional arrow displayed on a screen.

However, it is also possible to measure the various parameters of the cardiovascular system using a single pressure sensor unit, even without the use of a convex structure.

The use of a convex structure on which multiple pressure sensor units are distributed is advantageous. Ideally, these pressure sensor units cover the entire surface of the convex structure. This provides a choice of multiple pressure sensor units for measuring the various parameters of the cardiovascular system, which can in turn have multiple (e.g., two) measuring ranges. To determine which pressure sensor unit is in the optimum position above the artery, the signal or the measured values and/or conductances and/or resistances of all pressure sensor units are advantageously examined. These change over the course of a pulse wave. The optimally located pressure sensor unit and/or the m optimally located pressure sensor units is/are then characterized by the m highest amplitudes of the signal or of the measured values and/or conductances and/or resistances. In particular, the optimally located or the m optimally located pressure sensor unit(s) is/are used for implementing the (further) method, in particular for measuring blood pressure, arterial elasticity, the pulse wave, the pulse wave transit time, and the pulse wave velocity, and/or cardiac output and/or changes in cardiac output. In particular, m is equal to or greater than 2. Advantageously, the system is configured such that the optimally located or the m optimally located pressure sensor unit(s) is/are identified by the system, for example by a comparison of the highest or of the m highest amplitude(s) of the signal by means of the analysis unit, in which case, if no highest amplitude or fewer than m highest amplitudes is/are detected, and/or if a variation of at least 80%, in particular of 90%, more particularly of all amplitudes of less than 10%, in particular less than 5%, of the mean value of the amplitudes is detected, information that involves displacement operating instructions for correct positioning is presented on a display, for example on a smart device, e.g., by means of directional arrows on a screen.

In particular, the best measuring range is determined on the basis of the maximum value. Here, the smaller the measuring range, the more accurate the result.

Regardless of which variant is used for which application, the data from the pressure sensor unit(s) can be transmitted by means of a recording/processing/power and transmitting unit to an analysis unit and for the purpose of image or sound output.

The mobile solution, such as a smart device, for example a watch or smartphone, is preferred here.

The system can be equipped with a rechargeable battery or a battery, however, it would also be advantageous to ensure the availability of power via a smart device, for example a watch or a smartphone, or even within the wristband of fitness trackers.

Furthermore, the measuring sensor system can also be formed separately in the convex structure and can access external units (processing, wireless, etc.), for example a smart device.

This separation has advantages not only in terms of structural dimensions and the supply of power, as smart devices already have a developed operation and analysis infrastructure.

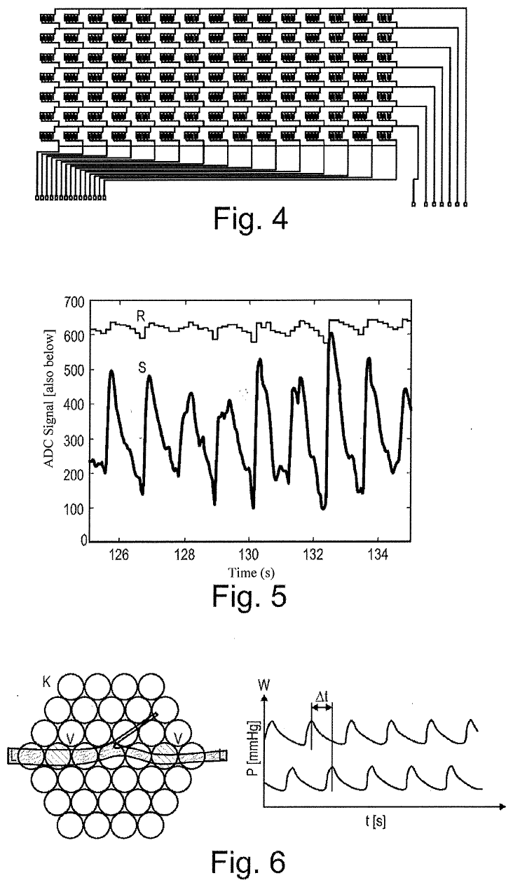

Crossover circuits of electrical conductor traces (see FIG. 4) are preferably used for the “shuttering” of data collection from the pressure sensor units, to enable faster readout of the pressure sensor units. With such an arrangement, which can also be described as tiled, the number of electrical conductor traces can be reduced, thus requiring less data to be processed and/or read out.

Blood pressure measurements are dependent upon the location of the measurement site in relation to the HIP (hydrostatic indifference point). As is further described in the patent, temporal, and spatial resolution relative to the HIP of the measurement site in the peripheral regions/extremities are advantageous for ascertaining the central continuous blood pressure.

When the living being, for example the human, is in a horizontal position, the location of the measurement site is irrelevant, since all measurable blood vessels are at roughly the same height in relation to the HIP.

Typically, measurements taken on the foot produce only minor changes with movement relative to the HIP.

To determine the course over time of the blood pressure within a pulse and over multiple pulses during physical movement, the height in relation to the HIP (hydrostatic indifference point) must be known. The blood pressure reading, e.g., in the arm, will change depending upon the height above the HIP. The change in blood pressure in the arm resulting from the change in height Δh is obtained as follows:

ΔP=αΔh

Here, α is an empirical measurement having a value of approximately α=1 mmHg/cm for the systolic blood pressure and approximately α=0.5 mmHg/cm for the diastolic blood pressure. If the current height of the arm is known and the blood pressure Pm in the arm can be measured, the central blood pressure Pz at the HIP can be calculated:

Pz=Pm−ΔP

The height of the arm can be determined using various technologies or devices. Possible known methods include determining the distance from a reference surface. This can be accomplished, for example, using an ultrasound distance sensor or using a laser range sensor. These sensors emit a signal (sound or laser pulse), which is reflected by a fixed reference surface, i.e., secured in place outside of the body. The length is determined from the transit time of the signal to the reference surface and back.

The current height and/or the change in the height of the measurement site, the at least one pressure sensor unit, and/or the system can also be determined using an acceleration sensor, a gyroscope, and/or an inertial sensor.

From the measured values of the acceleration sensor, the movement of the arm can be traced, and the current location of the arm can be determined, for example using the Velocity Verlet algorithm.

However, since even minute inaccuracies in acceleration measurement, such as can occur with today's acceleration sensors, can lead to major deviations in location or height, the sequence of movements in each step must be assessed accurately in order to determine the correct height. The procedure for this involves comparing the measured acceleration data with the anticipated acceleration data. For this purpose, known patterns of movement are compared with current acceleration data. If consistency is detected, movement to the current position can be ascertained.

In the simplest case, the current electrical resistance or conductance of a pressure sensor unit or of a measuring range of an SRS sensor or the resistance of a VRS sensor can occur when the pressure sensor unit is used in a voltage divider. The voltage, which drops across the pressure sensor unit, is the output measurement signal, which is in direct correlation to the conductance and/or resistance.

Alternatively or additionally, the pressure sensor unit can be read in another way. For this purpose the voltage, which drops across the pressure sensor unit, is amplified AC-coupled with a differential amplifier. The amplification is advantageously adjustable.

This signal changes with the slightest changes in the voltage that drops across the sensor, and thus with the slightest changes in pressurization. However, due to the AC coupling, the signal is independent of the actual pressurization.

The technical measures described in this example for receiving measurement signals can also be implemented differently. Other examples for receiving the non-amplified signal include, e.g., the use of a Wheatstone bridge, the constant current method for determining resistance, and installing the sensor in a resonant circuit. Many options for detecting these signals are known to those skilled in the art.

The voltages generated by these electronic assemblies are quantified, in particular, using a microcontroller, which in particular is part of the system, with the aid of an analog-to-digital converter. Depending upon the processing capacity of the microcontroller used, the quantified signals can either be analyzed directly or transmitted to an analysis unit. Advantageously, output data or calculated results are transmitted to an analysis unit or display unit by wireless means, e.g., with the Bluetooth standard.

Each measuring range of an SRS sensor covers a fixed force range. Advantageously, the force ranges overlap. During switching, it is not necessary for a fixed force value to be selected. Rather, two force values are selected for initiating the switch, in particular if the force ranges overlap, in order to generate and/or utilize a hysteresis of the switch. When the force is increased and the force value for shifting up in the current measuring range is exceeded, the system will switch to the next higher measuring range. In the higher measuring range, the force value for shifting down is then lower than the force value in the lower measuring range for shifting up. This prevents any unnecessary switching back and forth due to measurement noise when the applied force is within the range of the switching limits.

As described above, the electronic measuring system delivers two signals, in particular, which correspond to the base pressure S_{G}(t) and the mathematical time derivative of said pressure S_{D}′(t).

The base signals are first converted, in particular, by a calibration, dependent upon the sensor type, into the SI units N or N/s. Calibration can also take place in another unit, or can at least be based upon another unit, with such calibration advantageously being converted to the SI units.

The times or rates of the changes in the (electronically detected) signals vary. S_{G}(t) changes only gradually with a change in applied pressure or with a general change in blood pressure. In contrast, S_{D}′(t) changes continuously with the pulsatile pressure wave, and thus reflects the action of the heart. However, due to the electronics, the signal “forgets” changes in pressure over longer periods of time and thus always fluctuates around a zero value.

Mathematically, the current force, and from that also the pressure on the sensor F(t) at the time t is obtained using the following approximation, for example:

F(t)=S_{G}(t)+∫S_{D}′(t)dt

In this case t₀ is a point in time prior to the actual measurement, in which S_{D}′(t)=0 is true of all times t<t₀.

The signal S_{D}′(t) should change only for changes in pressure over small time scales; to ensure this, a running average S_{D}′(t) over the signal S_{D}′(t) is advantageously first determined.

The integral of the signal S_{D}′(t) as a sum is then approximated:

∫S_{D}′(t)dt≈Σ(S_{D}′(n)−S_{D}′(n))*Δt(n)

Here, n is the number of measured values since the start of measurement and Δt(n) is the interval of time between the measured values S_{D}′(n−1) and S_{D}′(n). The time t is given as t=ΣΔt(n).

Since errors occur with every numerical integration and since the measured values can also be noisy, implementation of the integral must be further optimized. The sum is therefore replaced with a recursive sum, and the result of the integral I(t) is obtained as:

$$I ( t ) \approx Σ ( {S}_{D}^{′} ( n ) - {S}_{D}^{′} ( n ) ) * Δ t ( n ) \approx \{ \begin{matrix}{I}_{0} = 0 \\ {I}_{n} = α {I}_{n - 1} + ( {S}_{D}^{′} ( n ) - {S}_{D}^{′} ( n ) ) * Δ t ( n )\end{matrix} \}$$

Here, for the attenuation factor, 0<α<1. This factor weakens the influence of older measurements and gives preference to the most recent values. This prevents calculation errors and measurement noise from accumulating. The factor must be empirically adapted to the type of sensor used.

Thus, the following results for the measured value of the pulsatile pressure wave P(t):

P(t)=β(S_{G}(t)+I(t))

Here, 1/β is the active area of the sensor.

If an array of multiple pressure sensor units is used to utilize the optimum position above an artery, advantageously all pressure sensor units are first read out over a period of several cardiac pulses. These readouts may be performed simultaneously or sequentially, depending on the capabilities of the measuring electronics. A simultaneous readout is preferred.

The best positioned pressure sensor unit(s) is/are characterized by its/their position directly above the artery. The amplitude of the pulsatile pressure wave is therefore at its maximum at this point. This/these pressure sensor unit(s) is/are advantageously used for further measurement and/or for implementation of the method according to the invention.

With a continuous measurement, detection of the best-positioned pressure sensor unit(s) is repeated at periodic time intervals.

The use of an acceleration sensor is advantageous. This sensor can be used to detect the physical movement of the person or animal being examined. If such movement exceeds a predefined limit, re-detection may be triggered.

The cardiac pulse is determined from the measured values of the pulsatile pressure wave. For this purpose, the measured value curve is analyzed for pronounced points. These may be the maxima or minima in the measurement wave or the measured values or in the pressure curve. The interval of time between two consecutive maxima or minima is the RR interval; the cardiac pulse expressed as number/minute is obtained from the following formula: 60s/RR interval in seconds.

In particular, the measured value wave or the profile of the pressure is examined on a running basis for minima and maxima. Various mathematical procedures are known to those skilled in the art.

A pulse wave velocity is obtained by measuring the pulsatile pressure wave at different points on the body. However, the points must be on a line between the heart and the point farthest from the heart.

This can be accomplished by two basic approaches. First, multiple, at least two, systems or assemblies and/or pressure sensor units according to the invention may be distributed on the body, or other instruments for determining the cardiac pulse may be used in addition to the assembly according to the invention. If external instruments are to be used, an open interface for the collected data and a real-time calculation of the data as a cardiac pulse-dependent measurement curve are required. For this purpose, for example, electrocardiogram (ECG) or plethysmography-based devices may be used, which are equipped with such open interfaces.

The measurement curves of the individual devices or of the individual sensors are examined for pronounced points. With the assembly according to the invention, these may be the maxima of the measured values of the pulsatile pressure wave.

The pronounced points of the various devices or sensors have a temporal offset from one another depending upon their location on the body. This temporal offset, divided by the distance between the measuring positions, gives the pulse wave velocity.

The use of a sensor array, in particular having a multiplicity of sensors as described above, has the advantages of easy operation and a simultaneous determination of blood pressure and pulse wave velocity.

To illustrate the use of a sensor array, FIG. 6, (K) shows one possible configuration of a sensor array. Beneath it, the pathway of an artery is shown (FIG. 6, (L)). If two sensors above the artery are selected (FIG. 6, (V)), the pulse wave velocity can be determined from the measurement curves of the two sensors (FIG. 6, (W)).

The respiratory rate can be measured and determined using various methods. For one, motion and acceleration sensors can measure the raising and lowering of the upper body and from this can determine the respiratory rate. Respiratory sinus arrhythmia (RSA) is a clear sign for determining the respiratory rate. The change in cardiac output is in direct correlation to the pulmonary/lesser circulatory system. All of the blood must pass through the lungs to absorb oxygen.

Furthermore, the use of acceleration sensors in an assembly according to the invention allows the actual performance to be determined. By determining the movement of individual body parts, the energy converted per unit of time, i.e., the mechanical performance, can be determined.

By comparing this theoretical performance obtained from the continuous measurement of cardiac output with the actual performance, the anticipated performance increase during physical exercise or during rehabilitation measures can be predicted.

Continuous monitoring of movement can also be helpful in medical applications. For example, if decreased cardiac output with an elevated heart rate is detected during the night, without any evidence of significant movement, the invention can draw diagnostic conclusions regarding the diseases in question.

Concentric diagrams are advantageously used as graphic depictions for users.

In 2D, 2.5D, or 3D, the totality of the measured data are displayed clearly. Conventional diagrams that have an X and a Y axis are confusing and difficult for users to comprehend due to the number of aspects to be displayed.

FIG. 1 shows an exemplary illustration of the measurement method. The pulsatile pressure wave (D), shown as a light gray curve, deforms the artery (L), which at rest has a constant thickness, between the two horizontal lines, which in most regions are parallel, steadily within the beat of the heart, distinguishable at the two sequential pressure maxima (G), which characterize the beginning and the end of a RR interval, for example, in which the artery (L) is deformed from its resting position, represented by the horizontal line (F). During said interval, the pressure wave fluctuates between the values for the diastolic blood pressure (minimum of the curve in which the artery has its resting position (B)) and the values for the systolic (A) blood pressure, the maxima of the gray curve. As a result of this, the pressure or the pulse pressure (N) is introduced via the artery surface (E) into the tissue (C) both below and above the artery, and continues to the surface of the skin (O).

The blood pressure is measured by an assembly (H) according to the invention and having the pressure sensor units (K) first being pressed against the skin (O) with an increasing level of pressure (J). For this purpose, the assembly according to the invention may be attached to a wristband (I).

FIG. 2 shows an exemplary diagram of a conventional wristband. A wristband of this type is equipped with a perforated band (I), with overhanging perforated band being held in place by a loop (P). The assembly according to the invention can be embodied in the form of this loop and can be used in place of said loop in the wristband. This has the advantage that, for one thing, the wristband is already located at a suitable site for blood pressure measurement, and for another, pressure can be exerted on the pressure sensor unit by adjusting the perforated band.

FIG. 3 shows, by way of example, a cross-section of one possible embodiment of the assembly of the invention for use on the wristband as an attachment. The assembly of the invention is divided into two parts. The pressure sensor units (K) arranged as an array on a convex structure underneath the wristband, and a processing/wireless and power unit (H) on top of the wristband. The wristband is passed through a slit (I). For measurement, a uniform or increasing pressure (J) is applied from above.

FIG. 4 shows an exemplary electrical circuit comprising multiple pressure sensor units (shown here as 7×15 sensors) in the crossover circuit. In this example, 15 circuit lines and 7 measuring lines are required.

FIG. 5 shows exemplary raw data from an assembly according to the invention. The pressure sensor units provide two measuring signals. An unamplified signal (R), which reflects the pressure on the pressure sensor unit, and an amplified signal (S), which reflects the changes in the pressure.

FIG. 6 shows a schematic and exemplary illustration of the measurement of pulse wave velocity by means of a sensor array (K). The two pressure sensor units (V) optimally lie above an artery (L). The two pressure sensor units (V) are used simultaneously for measurement. The measuring signals (W) are recorded. The transit time difference between the two measuring signals can be determined by analyzing the measuring signals for pronounced points. The pulse wave velocity is obtained by dividing the transit time difference by the distance between the two pressure sensor units (V).

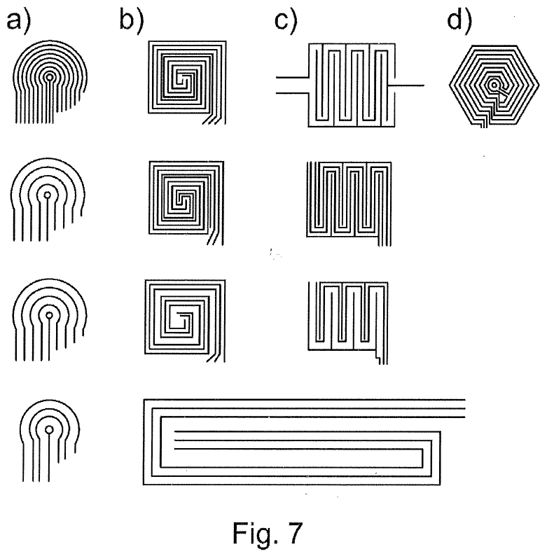

FIG. 7 shows examples of possible embodiments of the inventive configuration of the conductor trace arrays. In these, two conductor traces are always used together for conductance measurements, and different measuring ranges can be utilized depending upon which conductor trace pairs are used. In a), conductor trace arrays in a round configuration are shown, with any desired number of conductor trace arrays, depending upon the size. In b), another possible embodiment in the form of a square conductor trace array is shown, in which the conductor traces are generally in a spiral configuration. The first embodiment of a conductor trace array in column b has four conductor traces, while the second and third embodiments of conductor trace arrays from the top in column b) each feature a configuration having three conductor trace arrays. The embodiment of a conductor trace array that is third from the top in column b) features different distances between the conductor traces, and consequently has two measuring ranges with distinctly different measuring areas, defined by the corresponding conductor traces. The dimensions may also be adapted to the measurement task (see the embodiment of a conductor trace array that is fourth from the top in column b) and extends into column c)). The top three conductor trace arrays in column c) show different variants of meshing conductor trace arrays. Column d) shows, as a further possible embodiment of a conductor trace array, that the array of the conductor traces may also be more complex in configuration. This hexagonal shape of the conductor trace array is particularly suitable for covering a larger area, ideally without gaps.

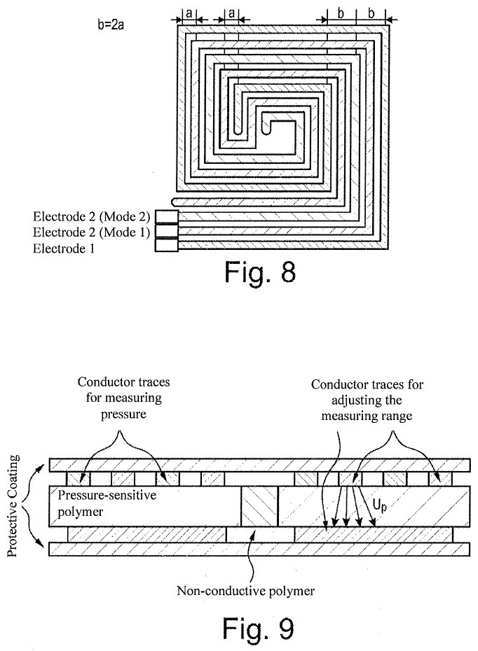

FIG. 8 shows a conductor trace array having three conductor traces, in which a first measuring range is selected, for example, in that measurement is performed between “electrode 1” and “electrode 2 (mode 1)”, and a second measuring range is selected in that measurement is performed between “electrode 1” and “electrode 2 (mode 2)”. Due to the changes in the distance between the conductor traces being used, and thus the distance to be bridged by the functional polymer, the conductance changes while the pressure remains the same. A greater distance results in a greater measuring range.

FIG. 9 shows a cross-section of two pressure sensor units arranged side by side and configured as VRS sensors. Shown in each pressure sensor unit are two conductor traces (light) for measuring conductance or pressure, arranged on a functional polymer. Additionally, a conductor trace (dark) is arranged on the functional polymer, and a conductive layer is arranged beneath the functional polymer, so that, when a voltage (Up) is applied between these, the measuring range can be influenced by altering the properties of the functional polymer.

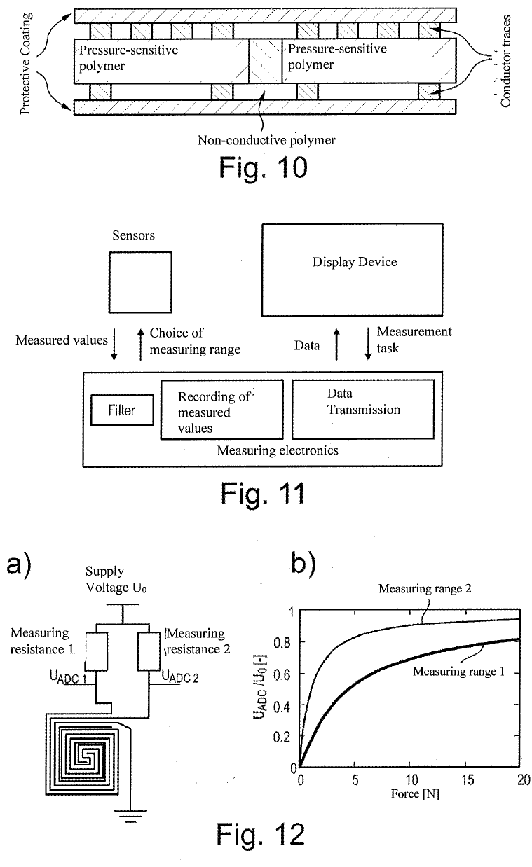

FIG. 10 shows a cross-section of two pressure sensor units arranged side by side. Above the functional polymer (also a pressure-sensitive polymer), each pressure sensor unit has two conductor trace arrays arranged a small distance from one another (due to the meandering configuration, each conductor trace array is visible twice in cross-section). Moreover, beneath the functional polymer (also a pressure-sensitive polymer) each pressure sensor unit has two conductor trace arrays separated from one another by a greater distance. The individual pressure sensor units are separated by a non-conductive polymer.

FIG. 11 shows an exemplary configuration of a measuring system for measuring conductances, and from these, pressures. The electrical signal for measuring the conductance is first processed by electronic filters. It is then digitized and sent to an analysis and display device. The display device, e.g., a smartphone, undertakes the analysis and display of the measured data. The display device of the electronic measuring system can also be used to implement various tasks, such as the selection of the conductor trace, a conductor trace region, the repetition rate, etc.

FIG. 12 is a schematic illustration showing one possible embodiment of the device according to the invention used as part of a voltage divider (a), along with a typical measurement curve of the conductances measured by such a device during a process in which pressure is applied. Due to the greater conductor trace spacing, measuring range 1 moves more slowly toward the maximum voltage than measuring range 2 with the narrower conductor trace spacing.

FIG. 13 is a schematic illustration showing one possible embodiment of the device according to the invention used as part of a voltage divider (a). The measuring range is adjusted by applying a voltage (U_{P}) across the polymer. In b), a typical measurement curve of the conductances with a change in voltage (U_{P}) is shown, which such a device measures during a process in which pressure is applied.

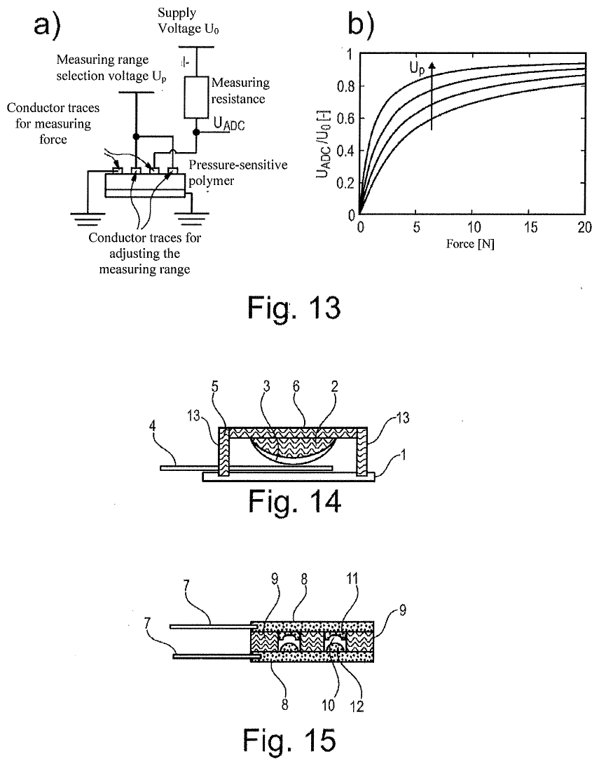

FIG. 14 shows a cross-section of a pressure sensor unit according to the invention. It comprises a substrate 1 on which conductor traces 4 are arranged, only one of which can be seen in this view. Said conductor traces are arranged, in particular, in the same manner as an array of FIG. 7 or 8. It also comprises a functional polymer 6, which has a conductive coating 3 made of a conductive polymer. It further includes a structural form 2 in the form of a spherical cap, and feet 13 with which it is placed in indentations 5 in the substrate 1. When pressure is applied from above and/or below, the functional polymer 6 deforms, in particular the feet 13 and the conductive coating 3 first contact the conductor traces, initially with a relatively high contact resistance, thereby connecting the conductor traces 4 to one another electrically. As the pressure is increased, the functional polymer 6 is deformed further. In particular, the feet 13 continue to deform and the curvature of the spherical cap 2 and of the conductive coating 3 becomes flattened, so that the contact surface area between the conductor traces 4 and the conductive coating 3 is enlarged. The contact resistance between the conductor traces 4 and the conductive coating 3 is further reduced as a result.

FIG. 15 shows a cross-section through another embodiment of a pressure sensor unit according to the invention. It has two metal conductors 7, each of which is fused into a conductive layer 8 of conductive polymer. Between the conductive layers 8 is an insulating layer 9 of an insulating polymer or lacquer. Said insulating layer has cavities 10. Within these cavities 10, the conductive layers 8 have roughly complementary extensions 11 and 12. When pressure is applied from above and/or below, the insulating layer 9 is compressed and the roughly complementary extensions 11 and 12 begin to touch at small points, lines, or surfaces. The resistance between the conductive layer 8 decreases and the conductance between them increases. As the pressure increases further, the insulating layer 9 is further compressed and the roughly complementary extensions 11 and 12 also deform further in the direction of a complementary shaping, so that their contact surface area is increased. This further reduces the contact resistance between the two conductive layers 8.

1. A system for the time-resolved measurement of blood pressure, arterial elasticity, a pulse wave transit time, a pulse wave velocity, a pulse wave, and/or a cardiac output and/or changes in the cardiac output, said system comprising:
at least one pressure sensor unit for a time-resolved pressure measurement of a pressure exerted by a pulse wave while said at least one pressure sensor unit is pressed against a patient's skin, wherein the at least one pressure sensor unit is configured to change at least one electrical conductance and/or resistance when subjected to the pressure;
wherein the at least one pressure sensor unit has:
at least two conductive layers and/or conductor trace arrays, in particular conductor trace networks, and
a functional polymer which is configured to be compressed when subjected to the pressure and to produce and/or change contact between the at least two conductive layers and/or conductor trace arrays; and/or
wherein the at least one pressure sensor unit is an air and/or gas pressure sensor and in particular has the at least two conductive layers with a dielectric arranged therebetween, and is configured such that, when subjected to the pressure, the dielectric becomes compressed and/or in particular a capacitance of the at least two conductive layers changes as a result; and
wherein the system further includes an actuator which is configured to press the at least one pressure sensor unit against the skin.

2. The system according to claim 1, wherein the at least one pressure sensor unit has at least one array of conductor traces and/or conductor trace networks of the at least two conductive layers and/or conductor traces, in particular conductor trace networks exposed, and a resistance-conductive and/or conductive polymer, which is pressed onto the at least one array of conductor traces and/or conductor trace networks when subjected to the pressure, and/or wherein the resistance-conductive and/or conductive polymer is at least one non-conductive polymer or a lacquer coating which has holes defined therein.

3. The system according to, claim 2, wherein the resistance-conductive and/or conductive polymer has a microstructure which deforms when subjected to the pressure and wherein a surface area of contact with the exposed at least one array of conductor traces and/or conductor trace networks increases, and the electrical contact improves, and in particular, electrical resistance between the at least one array of conductor traces and/or conductor trace networks and the resistance-conductive and/or conductive polymer and/or between conductor traces of the at least one array of conductor traces and/or conductor trace networks is reduced.

4. The system according to claim 2, wherein the resistance-conductive and/or conductive polymer is a part of the functional polymer, and wherein the functional polymer has a conductive surface formed by the resistance-conductive and/or conductive polymer.

5. The system according to claim 1, wherein the actuator is one of an electric actuator, a pneumatic actuator, and/or a hydraulic actuator, in particular including an electric vibration motor and/or an air bag, and when the actuator includes the air bag, the actuator is configured to inflate and/or to supply the air bag with air and for this purpose further includes a pump for pressing the at least one pressure sensor unit against the patient's body.

6. The system according to claim 1, wherein the system includes an air bag, in particular in the form of a cuff, and the at least one pressure sensor unit is positioned one of on the air bag, in the air bag, and/or in a volume fluidically connected to the air bag, and/or adjoining the volume or the air bag, and wherein the system is configured, in particular, such that the at least one pressure sensor unit detects the pressure exerted by the pulse wave while the air bag is being pressed against the skin, said pressure being transmitted through a gas in the air bag, and/or the pressure exerted by the pulse wave while the air bag is being pressed against the skin is transmitted through the air bag to the at least one pressure sensor unit.

7. The system according to claim 3, wherein when the at least one pressure sensor unit is in an idle state, only relatively few microstructural protrusions are in contact with the exposed at least one array of conductor traces and/or conductor trace networks, and the electrical resistance is between the resistance-conductive and/or conductive polymer and conductor traces of the exposed at least one array of conductor traces and/or conductor trace networks, and the pressure and/or a counterpressure causes the microstructure to deform, increasing an actual contact surface area of contact.

8. The system according to claim 1, wherein the at least one pressure sensor unit has a measuring range of at least 40 mmHg to at least 300 mmHg and/or a resolution of at least 0.5 mmHg, and/or is configured to take at least 1000 data values per second, and/or has a temporal resolution of at least 1 ms.

9. The system according to claim 1, wherein the system further comprises a calibration actuator, configured to press the at least one pressure sensor unit onto the skin with a known counterpressure, and/or comprises a counterpressure sensor for measuring a counterpressure with which the at least one pressure sensor unit is pressed against the skin.

10. The system according to claim 1, further comprising:
a calibration sensor, in particular a force and/or strain sensor and/or a strain gauge; and/or
a calibration actuator, which exerts pressure by way of a defined contraction; and/or
a vibration motor, in particular a motorized wristband.

11. The system according to claim 1, further comprising a counterpressure sensor for measuring a force with which the at least one pressure sensor unit is pressed onto the skin, proceeding from a finger of the patient, in particular simultaneously with measurements by the at least one pressure sensor unit, advantageously with at least 1000 measurements per second.

12. The system according to claim 1, wherein the at least one pressure unit comprises multiple pressure sensor units, in particular a sensor array comprised of a plurality of pressure sensor units, in particular as part of a sensitive sleeve, a sensitive surface, or an artificial skin, in particular of a robot.

13. The system according to claim 12, wherein the multiple pressure sensor units, in particular the sensor array comprised of the plurality of pressure sensor units, are arranged on a convex surface and/or a convex structure.

14. The system according to claim 1, further comprising an analysis unit for calculating systolic and/or diastolic blood pressure, arterial elasticity, the pulse wave transit time, the pulse wave velocity, the pulse wave, and/or the relative or absolute cardiac output from the measured values from the at least one pressure sensor unit, and in particular from a counterpressure sensor and/or a calibration sensor.

15. The system according to claim 1, wherein the at least one pressure sensor unit is no larger than a cherry pit, or 5 mm in diameter.

16. The system according to claim 1, further comprising at least one acceleration sensor and/or sensor for ascertaining a position/height relative to a hydrostatic indifference point (HIP), in particular an inertial sensor.

17. The system according to claim 1, further comprising a control unit and/or an analysis unit.

18. The system according to claim 1, configured to determine, from the at least one pressure sensor unit or a first set of pressure sensor units or from a plurality of pressure sensor units that is in an optimal position, and to pass on to the patient, information as to how the patient is able to readjust the positioning of the at least one pressure sensor unit or the first set of pressure sensor units or the plurality of pressure sensor units if said position does not meet a given requirement.

19. The system according to claim 1, configured for connection and/or coupling to at least one external measuring system, in particular an electrocardiogram (ECG) device or devices based on plethysmography, for determining a cardiac pulse, in particular for determining the pulse wave velocity, wherein the at least one external measuring system permits a real time measurement of a pulsatile pressure wave or ECG wave and is equipped with an open data interface that enables a real-time output of data.

20. A method for a time-resolved measurement of blood pressure, arterial elasticity, a pulse wave transit time, a pulse wave velocity, a pulse wave, and/or a cardiac output and/or changes in cardiac output by changing an electrical conductance and/or resistance and/or a capacitance between at least two conductive layers and/or between at least two conductor trace arrays, in particular conductor trace networks, by compressing a functional polymer and/or a dielectric by means of a pressure exerted by a pulse wave while said functional polymer and/or said dielectric is pressed against a patient's skin above an artery.

21. The method according to claim 20, wherein the at least two conductor trace arrays and the functional polymer are pressed against the skin with varying pressure and a resulting conductance and/or resistance is measured, and/or a change in the conductance and/or resistance is determined, in particular at least with a temporal resolution of 1 ms, wherein a varying pressure is increased, in particular, monotonically and/or continuously, in particular until with a further increase in a counterpressure and/or applied pressure, the pulse wave is not able to generate an increase in a measured conductance and/or a decrease in a measured resistance and/or pressure beyond a maximum measured conductance and/or a minimum measured resistance and/or pressure, wherein the applied pressure is applied in particular by inflating an air bag.

22. The method according to claim 21, wherein from the conductance and/or resistance and/or their change, the pressure and/or a change in the pressure is determined.

23. The method according to claim 21, wherein a systolic blood pressure is assumed to be the pressure at which, with a further increase in the counterpressure and/or applied pressure, the pulse wave cannot produce an increase in a measured pressure beyond a maximum measured pressure, and/or a diastolic blood pressure is assumed to be the pressure that corresponds to a minima of the measured values of the pulse wave, if the counterpressure and/or applied pressure is selected as the pressure, or higher than the pressure, at which the maximum measured pressure does not increase any further with increasing counterpressure and/or applied pressure.

24. The method according to claim 23, wherein the pressure of the applied pressure is then reduced to a value, in particular within a range of 1.5 times, in particular 1.3 times the systolic blood pressure, to a complete release of pressure.

25. The method according to claim 23, wherein the pressure of the applied pressure is subsequently reduced, and/or with a known first systolic blood pressure and/or a known first conductance and/or a first resistance of an at least one pressure sensor unit when subjected to the first systolic blood pressure, the counterpressure and/or applied pressure is reduced to less than 1.1 times the first systolic blood pressure or below the first systolic blood pressure, in particular to a level below the diastolic blood pressure, as long as a pulsatile pressure wave can be mapped, or is removed and ratios of the conductance and/or resistance then measured to the first conductance and/or the first resistance, and/or the ratios of the pressures associated with the conductance and/or resistance then measured to the first systolic blood pressure is used as a factor in determining a current blood pressure, a current arterial elasticity, a current pulse wave transit time, a current pulse wave velocity, a current pulse wave, and/or a current cardiac output and/or current changes in cardiac output from the first systolic blood pressure.

26. The method according to claim 25, wherein with the reduced applied pressure, continuous measurements of the conductance and/or of the pressure of the pulse wave are performed until a change in the pressure maxima of the pulsatile pressure wave is detected, in particular by more than 10%, and/or until a change in a distance between the pressure minima and the pressure maxima in the pulsatile pressure wave, in particular by more than 10%, and in particular the applied pressure is then decreased further and then increased again, in particular being increased monotonically and/or continuously, during which time the conductance is measured and/or the change in the conductance is determined, in particular at least with a temporal resolution of 1 ms.

27. The method according to claim 25, wherein the pulsatile pressure wave is measured using multiple sensors at different points on the patient's body, and from a temporal offset of the measurement curves relative to one another, the pulse wave transit time is determined, and in particular, a known distance between the multiple sensors is used to calculate the pulse wave velocity.

28. The method according to claim 20, wherein a change in cardiac output is determined by ascertaining a change in a value of the integral of all measured values, in particular of all measured conductance values and/or pressure values and/or pulse wave pressure values in the pulse wave, in particular between two systolic pressures and/or two diastolic pressures, and/or wherein the cardiac output is determined from the value of the integral of all measured values, in particular of all measured conductance values and/or pressure values and/or pulse wave pressure values in the pulse wave, in particular between two systolic pressures and/or two diastolic pressures multiplied by a cross-sectional area of an artery of the patient and/or of the patient's aortic arch.

29. The method according to claim 20, wherein the method is a method for continuous long-term monitoring.

30. The method according to claim 21, wherein the counterpressure and/or applied pressure is applied electrically, pneumatically, hydraulically, and/or manually, in particular by way of muscle contractions.

31. The method according to claim 20, wherein a height of a site where pressure is applied to the patient's skin in relation to a hydrostatic indifference point (HIP) is determined, and in particular, a correction of measured values is carried out, based upon the height of the site where pressure is applied to skin.

32. The method according to claim 20, wherein a data interface, in particular an open data interface, which enables a real-time output of data, of at least one, in particular external measuring system for determining a cardiac pulse, in particular an electrocardiogram (ECG) device or a plethysmography-based device, is used for determining the pulse wave velocity.

33. A use of a change in a capacitance, a conductance, and/or a resistance, between at least two conductive layers and/or between at least two conductor trace arrays, in particular conductor trace networks, resulting from a compression of a functional polymer and or a dielectric by means of a pressure exerted by a pulse wave while said functional polymer and/or dielectric is pressed against a patient's skin above an artery, for a time-resolved measurement of blood pressure, arterial elasticity, a pulse wave transit time, a pulse wave velocity, a pulse wave, and/or a cardiac output and/or changes in cardiac output.

34. The use according to claim 33, wherein a data interface, in particular an open data interface, which enables a real-time output of data, of at least one, in particular external measuring system for determining a cardiac pulse, in particular an electrocardiogram (ECG) device or a plethysmography-based device, is used for determining pulse wave velocity.
